(19) United States
(12) Patent Application Publication
(10) Pub. No.: US 2026/0124421 A1
(43) Pub. Date: May 7, 2026

(54) FIXATION STRUCTURE FOR A PATIENT INTERFACE

(71) Applicant: Fisher & Paykel Healthcare Limited, Auckland (NZ)

(72) Inventors: Michael Paul RONAYNE, Auckland (NZ); David Martin RUSSELL, Auckland (NZ); Philip Ian ROWE, Auckland (NZ); Gerard Charles CHESTERMAN, Auckland (NZ)

(21) Appl. No.: 19/118,954

(22) PCT Filed: Oct. 13, 2023
(86) PCT No.: PCT/IB2023/060328

§ 371 Date: Apr. 7, 2025

Related U.S. Application Data
(60) Provisional application No. 63/379,409, filed on Oct. 13, 2022.
(60) Provisional application No. 63/509,499, filed on Jun. 21, 2023.

(51) Int. Cl. A61M 16/06; A61M 16/00; A61M 16/04

(52) U.S. Cl. CPC A61M 16/0683; A61M 16/0003; A61M 16/04; A61M 16/0605; A61M 16/0672; A61M 2016/003; A61M 2202/0208; A61M 2205/3368

(57) ABSTRACT

There is provided a fixation structure for securing a patient interface to a patient. The fixation structure comprises a body having a first side and a second side. The fixation structure has a patient attachment so as to removably attach to a patient and a fixing element to removably attach to the patient interface. Sensor componentry is provided on or in one or more of the body, the patient attachment and the fixing element or attachable to one or more of the body, the patient attachment and the fixing element. There is also provided herein fixation structures comprising a fixation structure, systems for delivery of a breathable gas to a patient and patient monitoring systems.

### TECHNICAL FIELD

The present disclosure generally relates to patient interfaces and respiratory systems for providing breathable gases flow to a patient. More particularly, the present disclosure relates to respiratory support accessories and/or components with one or more sensors on or near the patient interface.

### BACKGROUND

Assisted breathing systems are available to aid patients in breathing for a number of reasons, for example due to, or in recovery from, a medical condition, during or following a medical procedure or otherwise for individuals who require a form of breathing support. In assisted breathing, respiratory gases are supplied to a patient through a flexible breathing tube. The gases expired by the patient may be channelled through a similar breathing tube or expelled to the patient's surroundings. The gases are, typically, administered to the patient through a patient interface, which may also comprise a short length of dedicated breathing tube to couple the interface with the supply tube. The patient interface may receive breathing gas from the flexible breathing tube and distribute it to the patient. Examples of a patient interface include a nasal cannula, nasal mask, oronasal or full face mask, and endotracheal (ET) tube.

When providing respiratory support to a patient, it can be beneficial to monitor one or more patient and/or respiratory support system parameters during the therapy. Parameter monitoring may detect interruptions to therapy or changes in patient condition requiring clinical intervention. In order to measure patient and/or system parameters, one or more sensors are used. These parameters can be used individually or in conjunction with further parameters in assessing the patient's health.

It would be desirable to provide an improved system, device or apparatus that overcomes or ameliorates one or more problem associated with the prior art or which provides a useful choice.

### SUMMARY

According to an aspect of the present disclosure there is provided a fixation structure for securing a patient interface to a patient, the fixation structure comprising: a body having a first side and a second side, in use the first side of the body facing towards the patient and the second side of the body facing away from the patient, wherein the first side of the body is removably attachable to the patient; a fixing element on the second side of the body, the fixing element is removably attachable to the patient interface; and sensor componentry provided on or in one or both of the body and the fixing element or attachable to one or both of the body and the fixing element.

According to an aspect of the present disclosure there is provided a fixation structure for securing a patient interface to a patient, the fixation structure comprising: a body having a first side and a second side; a patient attachment on the first side of the body; a fixing element on the second side of the body; and sensor componentry provided on or in any one or more of the body, the patient attachment and the fixing element or attachable to one or more of the body, the patient attachment and the fixing element.

As used herein, the term sensor componentry is used to describe a component or components that include at least one sensor. The sensor componentry may also include any further elements required to perform its function. For example, the sensor componentry may comprise any one or more of a printed circuit board (PCB), a wireless module, a substrate, an antenna and/or a power source.

In use, the first side of the body may face towards the patient, and the second side of the body may face towards the patient interface and/or away from the patient. The first side of the body may be removably attachable to the patient via the patient attachment. The fixation structure may secure the patient interface to the patient's skin. The fixation structure may be configured to secure the patient interface to the patient. The first side of the body may be removably attachable to the patient's skin via the patient attachment. The fixation structure may be configured to secure the patient interface to the patient's face.

A fixation structure may also be referred to as an adhesive patch, dermal patch or securement apparatus. Any of these terms may be used interchangeably herein.

In some configurations, the body comprises a substrate. The substrate may comprise a substrate material. The substrate may be a single layer or may have a plurality of layers. Each layer of the substrate may comprise the same material or a different material.

In some configurations, the sensor componentry is at least partially flexible. The sensor componentry may comprise a printed circuit board (PCB). The PCB may be flexible. The PCB may be at least partly flexible. The PCB may be provided on a polyurethane material base. The PCB may comprise a printed conductive ink. The PCB may be printed on the first side and/or second side of the body.

In some configurations, the PCB may comprise the sensor componentry. In other words, the PCB may include a plurality of features of which the sensor componentry is one.

In some configurations, the fixation structure comprises a patient attachment on the first side of the body. The patient attachment may be attached to the first side of the substrate. The patient attachment may be arranged to removably attach the fixation structure to the skin of the patient.

The patient attachment may comprise an adhesive. The patient attachment may be an adhesive layer.

The adhesive may be moisture permeable and/or porous. The adhesive may be dermatologically sensitive. The adhesive may be selected from any one or more of: a hydrocolloid-based adhesive material, a zinc oxide-based adhesive material, a silicone-based adhesive material, an acrylic-based adhesive material, such as a pressure sensitive adhesive, and/or a hydrogel-based adhesive material.

The adhesive may be non-conductive or conductive.

Electrical energy may be transferred to the sensor componentry from a power source, such as a battery and/or electrochemical cell. The power source may be a remote power source. Electrical energy may be transferred via the patient's skin with the patient acting as a conduit for the electrical energy. Such configurations may be effective where the patient attachment comprises a conductive adhesive to assist in transferring the electrical energy. The power source may supply electrical energy only when the fixation structure is attached to the patient. Attachment of the fixation structure to the patient may complete a circuit with the power source.

The sensor componentry may be located between the body and the patient attachment. Where the patient attachment is an adhesive, the sensor componentry may be located on a side of the body beneath or within the adhesive. The sensor componentry may be at least partly encapsulated within the patient attachment. The sensor componentry may contact the patient's skin directly. The patient attachment may comprise at least one aperture or localised thinned region. The aperture or localised thinned region may permit contact between the sensor componentry and the patient's skin or proximity between the sensor componentry and the patient's skin.

The fixing element may be a mechanical fastener. The fixing element may be mechanically configured to attach to the patient interface. The fixing element may comprise hook or loop connectors configured to engage with a complementary connection element on the patient interface. For example, the fixing element may comprise hook connectors and the patient interface comprise loop connectors, such that the hook and loop connectors may engage when brought together to attach the fixing element to the patient interface. Alternatively, the fixing element may comprise loop connectors and the patient interface comprise hook connectors, such that the hook and loop connectors may engage when brought together to attach the fixing element to the patient interface.

The sensor componentry may be located between the body and the fixing element. The sensor componentry may be on the body beneath the fixing element. The sensor componentry may be within the body.

The fixing element may be positioned in a first region of the second side of the body and the sensor componentry is positioned in a second region of the second side of the body. The second region may surround the first region of the second side of the body. Alternatively, the second region may surround the first region of the second side of the body.

The sensor componentry forms a unitary structure with the fixing element. The sensor componentry may be embedded within the fixing element. Embedding the sensor componentry within the fixing element may provide at least some additional physical protection for the sensor componentry.

The sensor componentry may comprise a plurality of components, one or more of which may be at least partially encapsulated within a waterproof housing.

The sensor componentry may be overmolded with a plastic material.

The waterproof housing may comprise at least one sheet of flexible material. The waterproof housing may comprise two sheets of flexible material. The sensor componentry may be positioned between the sheets of flexible material. The flexible material may comprise a hydrocolloid or other suitable material.

A lower power circuitry of the sensor componentry may not require encapsulation in a waterproof housing. In some configurations, a portion of the sensor componentry is encapsulated within a waterproof housing and another portion of the sensor componentry is not encapsulated within a waterproof housing.

The sensor componentry may be provided in or on a multi-layer arrangement. The body may comprise a plurality of layers. The layers of the body may form at least part of the multi-layer arrangement. Adjacent layers of the plurality of layers may be attached to one another by an adhesive.

The sensor componentry may be provided on a plurality of the layers of the body. The sensor componentry may comprise a plurality of PCBs as part of a multi-layer circuitry. The sensor componentry may comprise a PCB provided across a plurality of layers or as part of a multi-layer circuitry. The plurality of layers of the body may comprise a plurality of layers of the sensor componentry. The plurality of layers of the body may comprise a plurality of sensors or other elements of the sensor componentry. The sensor componentry may be split across the plurality of layers of the body.

The sensor componentry may transmit data or information. The data or information transmitted by the sensor componentry may be patient and/or respiratory support system parameter data. The sensor componentry may transmit the data or information to a remote location. The sensor componentry may transmit the data or information wirelessly. The sensor componentry may transmit the data or information via a wired connection.

The sensor componentry may receive data or information. The sensor componentry may receive the data or information from a remote location. The sensor componentry may receive the data or information wirelessly. The sensor componentry may receive the data or information via a wired connection.

As used herein the terms ‘data’ and ‘information’ may be used interchangeably. Any reference herein to ‘data’ may interchangeably refer to ‘information’ or to ‘data and information’ or ‘data or information’. Similarly, any reference herein to 'information may interchangeably refer to ‘data’ or ‘data and information’or ‘data or information’.

The fixation structure may comprise a wireless module. The fixation structure may comprise any one or more of an antenna, a receiver and a transceiver. The wireless module may comprise any one or more of an antenna, a receiver, a transceiver.

The fixation structure may comprise an antenna that transmits data sensed by the sensor componentry. The fixation structure may comprise a receiver to receive data. The fixation structure may comprise a transceiver to transmit and receive data. The sensor componentry may comprise the wireless module. The sensor componentry may comprise any one or more of an antenna, receiver and/or transceiver.

The fixation structure may comprise at least one inductive coil. The wireless module may comprise at least one inductive coil. In some configurations, the at least one inductive coil is provided on or in the body or fixing element.

The inductive coil may be oriented relative to an external magnetic field during use. The inductive coil may be at least partially oriented perpendicularly to the external magnetic field when the fixation structure is affixed to the patient. The external magnetic field may be generated by a magnetic field generator located in the proximity of the patient. The magnetic field generator may be on or in a mat or bed on which the patient lies.

The sensor componentry may be powered wirelessly. The sensor componentry may comprise a receiving coil that receives a power from an emitting coil. The sensor componentry may receive a power via the inductive coil.

The wireless module may be positioned adjacent to the sensor componentry. The sensor componentry may comprise the wireless module. The wireless module may comprise the sensor componentry.

The wireless module may transmit and/or receive data using a wireless protocol. The sensor componentry may transmit and/or receive data using a wireless protocol. The wireless protocol may comprise any one or more of Bluetooth, Wi-Fi or near-field communication (NFC). In some configurations, the data is transmitted at predetermined intervals.

The sensor componentry may transmit data at predetermined intervals. The wireless module may transmit data at predetermined intervals. The wireless module may be configured to receive a prompt from an external device. The wireless module may be configured to transmit data in response to said prompt. The wireless module may be interrogated or polled, being examples of a prompt, by an external device to cause the wireless module to transmit data.

The data sensed by the sensor componentry may be transmitted at predetermined intervals. The data sensed by the sensor componentry may be transmitted wirelessly or via wired connection. The sensor componentry may be configured to receive a prompt from an external device. The data sensed by the sensor componentry may be transmitted in response to the prompt. The sensor componentry may be interrogated or polled, being examples of a prompt, by an external device to cause data to be transmitted. The prompt may be received wirelessly. The prompt may be received via a wired connection. The external device may communicate with the sensor componentry wirelessly. The external device may communicate with the sensor componentry via a wired connection.

The sensor componentry may transmit patient identifier information. The patient identifier information may be transmitted wirelessly or via a wired connection. The patient identifier information may be sensed from a label on the patient. The label may comprise a machine readable code. The machine readable code may comprise a barcode or a QR code. The sensor componentry may comprise a memory that stores the patient identifier information. The wireless module may transmit patient identifier information. The wireless module may comprise a memory that stores the patient identifier information.

The sensor componentry may detect and/or retain usage information. For example, the sensor componentry may detect the duration of use of a patient interface device. Respiratory devices such as a patient interface may have a fixed or recommended usage period and the sensor componentry may be used to ensure that the device is not used beyond that period. For example, some interface devices may be recommended or authorised for use for a set period of days before needing to be replaced by a new interface. The usage duration information may be provided to or transmitted to a user device, which may enable a care giver to know when a device needs to be replaced.

The sensor componentry may be part of a system that gives an alarm, or other signal, when a usage period has expired and/or which informs the caregiver that the usage period has expired.

The sensor componentry may comprise a detector which determines when a patient interface is attached to the fixation structure. The sensor componentry may detect when a patient interface is removed from a fixation structure. The sensor componentry may detect when the fixation structure is attached to the skin of a patient. The sensor componentry may detect when an element, part or all of the sensor componentry is removed from the skin of a patient.

The wireless module may be powered via the same source of energy as the sensor componentry. The sensor componentry and the wireless module may comprise a unitary structure or be connected to one another. The wireless module may be powered via a different source of energy to the sensor componentry.

The sensor componentry may receive power from a battery. The battery may be a dry cell battery. The battery may be selected from any one of: a zinc-carbon cell, an alkaline cell, a lithium cell, a mercury cell, a silver-oxide cell, a nickel-cadmium cell, a lithium-ion cell or nickel-metal hydride cell. The battery may be a thin-film battery. The battery may be incorporated into the fixation structure. The battery may be adjacent to the sensor componentry. The battery may be adjacent to the wireless module. The battery may form part of the wireless module and/or the sensor componentry.

The sensor componentry may receive power from one or a plurality of sources. In an example, the sensor componentry may receive power wirelessly, such as via an inductive coil, but may also receive power from a fixed source, such as a battery. In this example, the sensor componentry may select using set instructions whether to pull power wirelessly or via the fixed source. When the induced current is above a threshold, the sensor componentry may draw power via an inductive coil. When the induced current is below a threshold, the sensor componentry may draw power from the fixed source, e.g. battery. When the inductive power is high enough, the battery may charge. This may ensure that there is sufficient source of energy when the inductive current is not high enough, for example when a patient is not located near to a transmitting coil. The battery may be used in accordance with an instruction protocol, such that when the battery charge is above a threshold the sensor componentry may draw power from it. When the battery charge is below a threshold, the sensor componentry may draw power from the inductive source. Otherwise, if one or both of the induced current and the battery charge are below threshold values the sensor componentry may signal that there is insufficient power, such as through an alarm signal. The sensor componentry may comprise a semi passive power source. In an example, the sensor componentry may comprise a battery assisted circuit where the battery is sleeping or offline until the circuit is interrogated/polled.

The sensor componentry may be powered by a surface mount battery or supercapacitor.

The sensor componentry may be powered by energy harvested from one or more of a solar, a piezoelectric, an electromagnetic or a thermal source.

The sensor componentry may be powered by a thermoelectric battery. The thermoelectric battery may utilise the Peltier effect to convert heat energy given off by the patient to electrical energy.

The sensor componentry may receive power from an electrochemical cell configured to convert chemical energy to electrical energy.

The electrochemical cell may comprise a fuel cell and/or a biofuel cell.

The electrochemical cell may comprise an enzymatic biofuel cell.

The electrochemical cell may be configured to generate electrical energy in the presence of a fuel, and at least one compound or composition. The at least one compound or composition may be a liquid, solid or gel. The at least one compound or composition may comprise one or more of an electrolyte and/or catalyst.

The electrochemical cell may comprise at least one enzyme selected at least in part based on the fuel.

The electrochemical cell may comprise at least one first enzyme and at least one second enzyme, wherein said at least one first enzyme and/or said at least one second enzyme are selected at least in part based on the fuel.

The fuel may comprise a hydrocarbon based fuel or substrate.

The fuel may comprise any one or more of: sugar, glucose, pyruvate, lactose, lactic acid, alcohol, (e.g. ethanol, methanol, glycerol), starch and/or mixture(s) thereof.

The fuel may comprise a naturally occurring molecule or biological substrate.

The electrochemical cell may comprise at least one reservoir comprising the fuel and configured to release the fuel contained therein.

The at least one reservoir may be configured to be breakable, pierceable, flexible and/or deformable. The electrochemical cell may comprise at least one membrane configured as a medium for facilitating electrochemical reactions.

The at least one membrane may be at least partially flexible and/or deformable. The at least one membrane may be liquid permeable. The at least one membrane may be porous. The at least one membrane may be conductive.

The electrochemical cell may comprise at least one protective layer. The at least one protective layer may comprise a solid cover, an at least partially flexible and/or deformable solid cover, and/or a coating. The at least one protective layer may at least partially cover the electrochemical cell. The at least one protective layer may be one or more of at least partially flexible, deformable, non-toxic, chemically stable, fluid-impermeable and/or electrically and/or temperature insulating.

The electrochemical cell may be incorporated into the fixation structure.

The electrochemical cell may be provided on or in one or more of the body, the patient attachment and the fixing element or attachable to one or more of the body, the patient attachment and the fixing element.

The electrochemical cell may be attachable to or integrally formed with the first side of the body, the second side of the body, the patient attachment and/or the fixing element, at least partly located between the body and the patient attachment, at least partly encapsulated within the patient attachment and/or at least partly located between the body and the fixing element.

The electrochemical cell may form a unitary structure with the fixing element and/or the biofuel cell may be embedded within the fixing element.

The electrochemical cell may be provided in or on a multi-layer arrangement. The electrochemical cell may comprise a plurality of layers and forms at least part of the multi-layer arrangement.

An electrochemical cell may be at least partially flexible and/or deformable so as to permit a corresponding flexing and deforming of the fixation structure. An electrochemical cell may be at least partially flexible and/or deformable so as to permit proximal or contacting attachment to surfaces of a patient's face.

The at least one cathode and/or at least one anode may be at least partially flexible and/or deformable.

In some examples, the at least one anode, at least one cathode, at least one membrane, at least one protective layer and/or at least one reservoir, may be integrally formed with any one or more of: the body, the first side of the body, the second side of the body, the substrate, the fixing element, the patient attachment, the adhesive layer thereof, the sensor componentry, the printed circuit board thereof and/or any other features of the fixation structure described herein.

The electrochemical cell(s) described herein may be employed as a fixed source of power e.g., in a manner analogous to how example battery described herein are employed as a fixed source of power.

The body may at least in part comprise a polymer, such as polymer resin, polymer plastic and/or composite. The polymer may be thermoplastic. The body may comprise a polyurethane material. The polyurethane material may comprise a thermoplastic polyurethane

The body may at least in part be formed from an at least partially porous material. In an example, at least one layer of the body may be formed from the at least partially porous material.

At least part of the fixation structure may be breathable.

The body may at least partly be formed from a material with a moisture vapour transmission rate (MVTR) >0 g/m²/24 h. The MTVR may be above about 100, 200, 400, 1000, 1500, 2000, 5000 or 10,000g/m²/24 h.

The body may be formed at least in part from a thermoplastic or thermosetting polymer. The polymer may be a polyimide. The polymer may be a film. At least one layer of the body may be formed from a film, such as a polyimide film.

In some configurations, the sensor componentry can sense at least one parameter of the patient. The sensed parameter may be selected from any one or more of: temperature, skin colour, patient movement, CO₂ concentration in the patient's blood (PCO₂), saturation of peripheral oxygen (SPO₂), heart rate, heart rhythm, heart rate variability, electrical activity and/or respiratory rate.

The sensor componentry may comprise a passive sensor.

The sensor componentry may comprise a temperature sensor. The temperature sensor may measure a body temperature of the patient. The temperature sensor may comprise any one or more of a thermistor, a resistance temperature detector (RTD), electrodermal activity (EDA) sensor, pyroelectric detector or a thermocouple.

The temperature sensor may be flexible.

The temperature sensor may be a passive temperature sensor. The passive temperature sensor may be a thermistor which requires a current applied to measure the voltage. The temperature sensor may be a thermocouple that measures the temperature in dependence on the voltage between two different electrical conductors, e.g. two different metals.

The sensor componentry may comprise a colour sensor. The colour sensor may be configured to detect a colour of the skin of the patient. The sensor componentry may comprise any one of a PCO₂, SPO₂ or pulse oximeter sensor that measures the colour of skin. The sensor componentry may detect one or both of respiratory rate and colour of the skin.

The sensor componentry may comprise at least one film that changes colour in response to a change in a parameter of the patient. The sensor componentry may comprise a colour sensor configured to sense the colour of the film. The film may be a thermochromic film that changes colour in response to a change of temperature of the patient. The film may, for example, change colour to reflect a detected CO₂ concentration in the patient's blood (PCO₂).

A PCO₂ sensor may measure the parameter precisely or approximately. The sensor componentry may be configured to measure abrupt variations in PCO₂. An alarm may be triggered if the variation in the PCO₂ value is above or below a set threshold. The sensor componentry may measure the partial pressure of venous carbon dioxide (PvCO₂). This measurement may be taken remotely and/or wirelessly.

The sensor componentry may comprise a motion sensor. The motion sensor may comprise a microelectromechanical system (MEMS) accelerometer. The motion sensor may detect any movement of the patient. The motion sensor may detect a breathing motion or breathing action of the patient.

The sensor componentry may comprise an electrocardiogram (ECG) sensor.

The sensor componentry may comprise a pulse oximeter sensor. The pulse oximeter sensor may determine at least a saturation of peripheral oxygen (SPO₂) in the blood of the patient.

One or more signal processing components may be disposed on the fixation structure. The signal processing component(s) may be provided on one or both of the first side and second side of the body. For example, one or more signal processing components may be disposed on either side of the PCB. Alternatively, signal processing may be performed externally to the patient interface, such as by a patient monitor, a respiratory therapy device, or other external device.

In some configurations, the sensor componentry comprises at least one light source. The light source may be a photodiode or LED. The sensor componentry may comprise a photoplethysmography (PPG) sensor. The PPG sensor may utilise the light source and a photodetector. The PPG sensor may make an optical measurement of the arterial volume using the light source. The PPG sensor may be used to measure a blood flow in the patient.

In some configurations, the fixation structure may comprise an aperture. A sensor of the sensor componentry may be located in or adjacent to the aperture. The sensor may sense a parameter through the skin of the patient when the fixation structure is attached to the patient. The sensor may sense a parameter of the patient when the fixation structure is attached to the patient.

In some configurations, the patient fixation structure may comprise a thinned region. Any one or more of the body, patient attachment and/or fixing element may comprise a thinned region. As an example, where the patient attachment comprises an adhesive, the layer of adhesive may be thinned in the thinned region. A sensor of the sensor componentry may be located adjacent to or on the thinned region(s). The sensor may sense a parameter of the patient through the thinned region(s) when the fixation structure is attached to the patient. The sensor may be a colour sensor or pulse oximeter sensor.

In some configurations, the fixation structure may be formed from a plurality of layers. The body of the fixation structure may comprise at least two separate layers. A first layer may comprise the patient attachment and a second layer may comprise the fixing element. The first and second layers may be removably attachable to one another.

The sensor componentry may be placed in between the first and second layers such that it is retained in its position within the body between the first and second layers when they are attached together.

One or both of the first layer and second layer may itself comprise a plurality of layers. The first and second layers may attach to one another by an adhesive. A side of the first layer may comprise the adhesive and/or a side of the second layer may comprise the adhesive. The sensor componentry may be provided on or within one or more of the plurality of layers of the body.

Another aspect of the present disclosure provides a fixation structure for securing a patient interface to a patient, the fixation structure comprising: a body comprising a main region and a first extension, each of the main region and the first extension having a first side and a second side, wherein the first side of the main region is removably attachable to the patient; a first fixing element on the first extension, the first fixing element is removably attachable to the patient interface; and a first sensor componentry provided on or in one or both of the body, and the first fixing element or attachable to one or both of the body and the first fixing element, wherein the first extension is attachable to the second side of the main region.

According to an aspect of the present disclosure there is provided a fixation structure for securing a patient interface to a patient, the fixation structure comprising: a body comprising a main region and a first extension, each of the main region and the first extension having a first side and a second side; a first fixing element on the first extension; and a first sensor componentry provided on or in one or both of the body, and the first fixing element or attachable to one or both of the body and the first fixing element.

The first extension may be attachable to the second side of the main region.

The first side of the main region may comprise a patient attachment. The first side of the main region may be removably attachable to the patient via the patient attachment.

In use, the first side of the body may face towards the patient, and the second side of the body may face towards the patient interface and/or away from the patient. The first side of the body may be removably attachable to the patient via the patient attachment.

The body may comprise a substrate. The substrate may comprise a substrate material. The substrate may be a single layer or may have a plurality of layers. Each layer of the substrate may comprise the same material or a different material. The substrate may be flexible.

The first fixing element may be on the first side of the first extension. The second side of the main region and second side of the first extension may be attachable to one another. The first extension may be pivotable relative to the main region from a first orientation to a second orientation where the second side of the first extension attaches to the second side of the main region. In the first orientation, the main region and first extension may be substantially adjacent and parallel to one another. In the second orientation, the first extension may be folded back on top of the main body with the second sides of each in contact.

The body may comprise a second extension having a first side and a second side. The first extension may be attached to a first edge of the main region and the second extension may be attached to a second edge of the main region. The first edge and second edge of the main region may be substantially parallel to one another. The first edge and the second edge of the main region may be on opposed sides of the main region from one another. The first edge of the main region may form a hinge about which the first extension is pivotable relative to the main region. The second edge of the main region may form a hinge about which the second extension is pivotable relative to the main region.

A second fixing element may be on the first side of the second extension. The second fixing element may be removably attachable to the patient interface. The second fixing element may be the same type of fixing element as the first fixing element. For example, the first and second fixing elements may both comprise hook connections or loop connections, each intended to attach to a complementary connection on the patient interface.

The second side of the main region and the second side of the second extension may be attachable to one another. The second extension may be pivotable relative to the main region from a first orientation to a second orientation where the second side of the second extension attaches to the second side of the main region. In the first orientation the main region and second extension may be substantially adjacent and parallel to one another. In the second orientation, the second extension may be folded back on top of the main body with the second sides of each in contact. The fixation structure may be configured such that only one of the first extension and the second extension may pivot relative to and attach to the main region at a time. The other of the first extension and the second extension may be removed from main region and may be attached to a patient's skin at a different location.

One or both of the first extension and the second extension may be separable from the main region. In some configurations, during use, only one of the extensions may be detached from the main region. The second side of the separated first extension or the second side of the separated second extension may be removably attachable to the patient. In other words, after separation from the main region, the first extension or second extension may be separately attached to a patient. In that case, there may be two parts to be attached to the patient separately, namely a first part that is the main region with either of the first or second extension attached to it, and a second part that is the other of the first of the extension or second extension that has been separated from the main region.

One or both of the second side of the main region and the second side of the first extension may comprise an attachment. One or both of the second side of the main region and the second side of the second extension may comprise an attachment. Each of the second side of the first extension and second side of the second extension may comprise an attachment. Each of the second side of the main region, second side of the first extension and second side of the second extension may comprise an attachment. The first side and second side of the main region may comprise an attachment.

At least one said attachment may comprise an adhesive. The adhesive may be non-conductive or conductive. The adhesive may be moisture permeable. The adhesive may be porous. The adhesive may be dermatologically sensitive. The adhesive may be selected from any one or more of: a hydrocolloid-based adhesive material, a zinc oxide-based adhesive material, a silicone-based adhesive material, and/or a hydrogel-based adhesive material.

The first sensor componentry may be at least partially flexible. The first sensor componentry may be flexible.

The fixation structure may comprise at least one PCB. The first sensor componentry may comprise a PCB. The PCB may be flexible. The PCB may be provided on a polymer material base, such as a polyurethane. The PCB may comprise a printed conductive ink. The PCB may be printed directly onto the body. The PCB may be printed directly onto the main region of the body. The PCB may be printed directly onto the first side of the main region of the body. The PCB may be printed directly onto the second side of the main region of the body. The PCB may be printed directly onto a fixing element. The PCB may be printed directly onto another element of the fixation structure. The PCB may be printed on the first side of the body. The PCB may be printed on the second side of the body. The fixation structure may comprise a plurality of PCBs. The PCB may comprise one or more chips or microchips. The PCB may comprise an individual sensor. The PCB may comprise a plurality of sensors. Each PCB may be selected to include any one or more of the PCB features described herein.

The fixation structure may comprise a patient attachment on the first side of the body. The patient attachment may be attached to the first side of the substrate. The patient attachment may be configured to removably attach the fixation structure to the skin of the patient.

The first side of the main region of the body may comprise a patient attachment. The patient attachment may at least in part comprise an adhesive. The adhesive may be non-conductive or conductive.

Electrical energy may be transferred to the first sensor componentry from a power source, such as a battery. The power source may be a remote power source. Electrical energy may be transferred via the patient's skin with the patient acting as a conduit for the electrical energy. Such configurations may be effective where the patient attachment comprises a conductive adhesive to assist in transferring the electrical energy. The power source may supply electrical energy only when the fixation structure is attached to the patient. Attachment of the fixation structure to the patient may complete a circuit to activate the remote battery.

The adhesive may be moisture permeable and/or porous. The adhesive may be dermatologically sensitive. The adhesive may be selected from any one or more of: a hydrocolloid-based adhesive material, a zinc oxide-based adhesive material, a silicone-based adhesive material, and/or a hydrogel-based adhesive material.

The first sensor componentry may be located between the body and the patient attachment. Where the patient attachment is an adhesive, the first sensor componentry may be located on a side of the body beneath or within the adhesive. The first sensor componentry may be at least partly encapsulated within the patient attachment.

The first sensor componentry may be located between the body and the fixing element. The first sensor componentry may be on the body beneath the fixing element. The first sensor componentry may be within the body.

The fixing element may be positioned in a first region of the second side of the body. The first sensor componentry may be positioned in a second region of the second side of the body. The second region may surround the first region of the second side of the body.

The sensor componentry may form a unitary structure with the fixing element. The sensor componentry may be at least partly embedded within the fixing element.

The sensor componentry may be at least partly encapsulated within a waterproof housing. The sensor componentry may be overmolded with a polymer or plastic material.

The first sensor componentry may be located between the main region and the patient attachment. Where the patient attachment is an adhesive, the sensor componentry may be located on a side of the main attachment beneath or within the adhesive. The sensor componentry may be encapsulated within the patient attachment.

The first sensor componentry may be located between the first extension and the main region when the first extension attaches to the second side of the main region. The first sensor componentry may be separate from the body when the first extension and the main region are not attached to one another and becomes attached between the first extension and main region when they are attached together. Alternatively, the first sensor componentry may be provided on one or both of the first extension and main region prior to their attachment together. The first sensor componentry may be sandwiched between the first extension and the main region when the first extension and main region are attached together.

The first sensor componentry may be located on the first extension. The first sensor componentry may be located on one or both of the first side and second side of the first extension. At least a part of first sensor componentry may be printed on the first extension. At least a part of the first sensor componentry may be printed on one or both of the first side and the second side of the first extension. The PCB may be printed on the first side of the first extension. The PCB may be printed on the second side of the first extension. In some configurations, the PCB is printed on both of the first side and the second side of the first extension.

The first sensor componentry may be located between the first extension and the attachment of the first extension. Where the attachment of the first extension is an adhesive, the first sensor componentry may be located on a side of the first extension beneath or within the adhesive. The first sensor componentry may be at least partially encapsulated within the attachment of the first extension.

The first sensor componentry may be located between the first extension and the fixing element.

The first sensor componentry may be located between the second extension and the main region when the second extension attaches to the second side of the main region. The first sensor componentry may be separate from the body when the second extension and the main region are not attached to one another and becomes attached between the second extension and main region when they are attached together. Alternatively, the first sensor componentry may be provided on one or both of the second extension and main region prior to their attachment together. In some configurations, the first sensor componentry is sandwiched between the second extension and the main region when the second extension and main region are attached together.

The first sensor componentry may be located on the second extension. The first sensor componentry may be located on one or both of the first side and second side of the second extension. At least a part of first sensor componentry may be printed on the second extension. At least a part of the first sensor componentry may be printed on one or both of the first side and the second side of the second extension. According to configurations, the PCB is printed on the first side of the second extension. The PCB may be printed on the second side of the second extension. The PCB may be printed on both of the first side and the second side of the second extension.

The first sensor componentry may be located between the second extension and the attachment of the second extension. Where the attachment of the second extension is an adhesive, the first sensor componentry may be located on a side of the second extension beneath or within the adhesive. In some configurations, the first sensor componentry is at least partially encapsulated within the attachment of the second extension.

The first sensor componentry may be located between the second extension and the fixing element.

The first sensor componentry may be located on both of the first extension and the second extension. The first sensor componentry may be located on each of the first extension and second extension in any manner as described above in relation to either of the first or second extensions. In some configurations, the first sensor componentry is located on two or more of the first extension, the second extension, and/or the main region of the body. In some configurations, the first sensor componentry is located on each of the first extension, the second extension, and the main region of the body.

The sensor componentry may comprise one or more sensors. The sensor componentry may take readings of the same parameter from a plurality of locations to obtain a more accurate reading of that parameter.

In some configurations, the fixation structure comprises only a first sensor componentry. The fixation structure may comprise a plurality of sensors. Each sensor of the plurality of sensors may be part of the same sensor componentry.

The fixation structure may comprise a second sensor componentry. The second sensor componentry may be located adjacent to or distal to the first sensor componentry. The second sensor componentry may be located in any location on a fixation structure or attached in any position to a fixation structure as described herein in relation to the first sensor componentry. The second sensor componentry may be attached or located in the same location as the first sensor componentry. The second sensor componentry may be attached or located in a different location to the first sensor componentry.

The second sensor componentry may be located on the second extension. The second sensor componentry may be located between the second extension and the attachment of the second extension. Where the attachment of the second extension is an adhesive, the second sensor componentry may be located on a side of the second extension beneath or within the adhesive. In some configurations, the second sensor componentry is at least partly encapsulated within the attachment of the second extension.

The second sensor componentry may be located between the second extension and the fixing element.

The first sensor componentry and second sensor componentry may comprise a sensing circuit. The first sensor componentry and second sensor componentry may sense the same, different or linked parameters. Use of the first sensor componentry and second sensor componentry may enable a more accurate reading of one or a plurality of parameters. Use of the first sensor componentry and second sensor componentry may enable detection of measurement errors. Use of the first sensor componentry and/or second sensor componentry may enable detection of a faulty sensor componentry. Use of the first sensor componentry and/or second sensor componentry may enable detection of an incorrectly placed sensor componentry.

The first and/or second sensor componentry may be provided in or on a multi-layer arrangement. The body may comprise a plurality of layers. The layers of the body may form at least part of the multi-layer arrangement. Adjacent layers of the plurality of layers may be attached to one another by an adhesive.

The first and/or second sensor componentry may be provided on a plurality of the layers of the body.

The first and/or second sensor componentry may comprise a plurality of PCBs as part of a multi-layer circuitry.

The first and/or second sensor componentry may transmit data or information. The data or information transmitted by the first and/or second sensor componentry may be patient and/or respiratory support system parameter data. The first and/or second sensor componentry may transmit the data or information to a remote location. The first and/or second sensor componentry may transmit the data or information wirelessly or via a wired connection.

The first and/or second sensor componentry may receive data or information. The first and/or second sensor componentry may receive the data or information from a remote location. The first and/or second sensor componentry may receive the data or information wirelessly or via a wired connection.

The fixation structure may comprise a wireless module. The fixation structure may comprise any one or more of an antenna, a receiver, and a transceiver. The wireless module may comprise any one or more of an antenna, a receiver, a transceiver.

The fixation structure may comprise an antenna that transmits data sensed by the sensor componentry. The fixation structure may comprise a receiver to receive data. The fixation structure may comprise a transceiver to transmit and receive data. The wireless module may comprise any one or more of the antenna, receiver or transceiver. The sensor componentry may comprise the wireless module. The sensor componentry may comprise any one or more of an antenna, receiver and/or transceiver.

The fixation structure may comprise at least one inductive coil. The wireless module may comprise at least one inductive coil. In some configurations, the at least one inductive coil is provided on or in the body. In some configurations, the at least one inductive coil is provided on or in the fixing element. In some configurations, the at least one inductive coil is provided on or in the patient attachment. The first and/or second sensor componentry may comprise at least one inductive coil. The at least one inductive coil may be provided on or in one or more of the first extension, the second extension or the main region.

The wireless module may be positioned adjacent to the first and/or second sensor componentry. The first and/or second sensor componentry may comprise the wireless module. The wireless module may comprise the first and/or second sensor componentry.

The wireless module may transmit and/or receive data using a wireless protocol. The first and/or second sensor componentry may transmit and/or receive data using a wireless protocol. The wireless protocol may comprise any one or more of Bluetooth, Wi-Fi or near-field communication (NFC). The data may be transmitted at predetermined intervals.

The first and/or second sensor componentry may transmit data at predetermined intervals. The wireless module may transmit data at predetermined intervals.

The wireless module may be configured to receive a prompt from an external device. The wireless module may be configured to transmit data in response to said prompt. The wireless module may be interrogated or polled by an external device to cause the wireless module to transmit data.

The data sensed by the first and/or second sensor componentry may be transmitted at predetermined intervals. The sensor componentry may be configured to receive a prompt. The data sensed by the first and/or second sensor componentry may be transmitted in response to the prompt. The prompt may be received wirelessly. The prompt may be received via a wired connection. The first and/or second sensor componentry may be interrogated or polled by an external device to cause data to be transmitted. The external device may communicate with the first and/or second sensor componentry wirelessly or via a wired connection.

The first and/or second sensor componentry may transmit patient identifier information. The patient identifier information may be transmitted wirelessly or via a wired connection. The patient identifier information may be sensed from a label on the patient. The label may comprise a machine readable code. The machine readable code may comprise a barcode or a QR code. The first and/or second sensor componentry may comprise a memory that stores the patient identifier information. The wireless module may transmit patient identifier information. The wireless module may comprise a memory that stores the patient identifier information.

The inductive coil may be oriented relative to an external magnetic field during use. The inductive coil may be at least partially oriented perpendicularly to the external magnetic field when the fixation structure is affixed to the patient. The external magnetic field may be generated by a magnetic field generator located in the proximity of the patient.

The first and/or second sensor componentry may be powered wirelessly. The first and/or second sensor componentry may comprise a receiving coil that receives a power from an emitting coil. The first and/or second sensor componentry may receive a power via the inductive coil.

The wireless module may be powered via the same source of energy as the first and/or second sensor componentry. The first and/or second sensor componentry and the wireless module may comprise a unitary structure or be connected to one another. In some configurations, the wireless module is powered via a different source of energy to the first and/or second sensor componentry.

The first and/or second sensor componentry may receive power from a battery. The battery may be a dry cell battery. The battery may be selected from any one of: a zinc-carbon cell, an alkaline cell, a lithium cell, a mercury cell, a silver-oxide cell, a nickel-cadmium cell a lithium-ion cell or nickel-metal hydride cell. The battery may be a thin-film battery. The battery may be incorporated into the fixation structure. The battery may be adjacent to the first and/or second sensor componentry. The battery may be adjacent to the wireless module. The battery may form part of the wireless module and/or the first and/or second sensor componentry.

The first and/or second sensor componentry may receive power from an electrochemical cell configured to convert chemical energy to electrical energy and/or may be powered by energy harvested from an electrochemical source. The electrochemical cell may comprise a fuel cell, a biofuel cell and/or an enzymatic biofuel cell configured to generate electrical energy in the presence of a fuel. The electrochemical cell may comprise any one or more features as disclosed herein in relation to any other aspect, embodiment or configuration or example of the present disclosure.

The first and/or second sensor componentry may receive power from one source or a plurality of sources. For example, the first and/or second sensor componentry may receive power wirelessly, such as via an inductive coil, but may also receive power from a fixed source, such as a battery and/or electrochemical cell.

In this example, the first and/or second sensor componentry may select using set instructions whether to pull power wirelessly or via the fixed source. When the induced current is above a threshold, the first and/or second sensor componentry may draw power via an inductive coil. When the induced current is below a threshold, the first and/or second sensor componentry may draw power from the fixed source, e.g. battery.

When the inductive power is high enough, the battery may charge itself. This may ensure that there is sufficient source of energy when the inductive current is not high enough, for example when a patient is not located near to a transmitting coil. The battery may be used in accordance with an instruction protocol, such that when the battery charge is above a threshold the first and/or second sensor componentry may draw power from it. When the battery charge is below a threshold, the first and/or second sensor componentry may draw power from the inductive source. Otherwise, if one or both of the induced current and the battery charge are below threshold values the first and/or second sensor componentry may signal that there is insufficient power, such as through an alarm signal.

The first and/or second sensor componentry may comprise a semi passive power source. For example, the first and/or second sensor componentry may comprise a battery assisted circuit where the battery is sleeping or offline until the circuit is interrogated/polled. The use of a semi passive power source may provide a compromise between purely passive circuits that have no battery and active circuits which contain a battery that are used continuously.

Where the fixation structure comprises a plurality of sensors each of the sensors may be powered via the same energy source or via different energy sources. Each of the plurality of sensors may be powered via a respective one of a plurality of batteries. Each sensor of the plurality of sensors may be part of the same sensor componentry.

The first and/or second sensor componentry may be powered by a surface mount battery or supercapacitor. The first and/or second sensor componentry may be powered by energy harvested from one or more of a solar, a piezoelectric or a thermal source. The first and/or second sensor componentry may be powered by a thermoelectric battery. The thermoelectric battery may utilise the Peltier effect to convert heat energy given off by the patient to electrical energy.

The body may at least in part comprise a polymer, such as polymer resin, polymer plastic and/or composite. The polymer may be thermoplastic. The body may at least in part comprise a polyurethane material. The polyurethane material may comprise a thermoplastic polyurethane.

The body may at least in part be formed from an at least partially porous material. At least one layer of the body may be formed from the at least partially porous material.

At least part of the fixation structure may be breathable.

The body may at least partly be formed from a material with a moisture vapour transmission rate (MVTR) >0 g/m²/24 h. The MTVR may be above about 100, 200, 400, 1000, 1500, 2000, 5000 or 10,000 g/m² per day.

The body may be formed from a polyimide film. In some configurations, at least a part of the body is formed from a polyimide film. In some configurations, at least one layer of the body is formed from a polyimide film.

The fixing element may be a mechanical fastener. The fixing element may be configured to attach to the patient interface. The fixing element may comprise hook or loop connectors configured to engage with a complementary connection element on the patient interface. For example, the fixing element may comprise hook connectors and the patient interface comprises loop connectors, such that the hook and loop connectors may engage when brought together to attach the fixing element to the patient interface. Alternatively, the fixing element may comprise loop connectors and the patient interface comprises hook connectors, such that the hook and loop connectors may engage when brought together to attach the fixing element to the patient interface.

In some configurations, the first and/or second sensor componentry can sense at least one parameter of the patient. The sensed parameter may be selected from any one or more of: temperature, skin colour, patient movement, CO₂ concentration in the patient's blood (PCO₂), saturation of peripheral oxygen (SPO₂), heart rate, heart rhythm, heart rate variability, electrical activity and/or respiratory rate.

In some configurations, the first and/or second sensor componentry comprises a passive sensor.

The first and/or second sensor componentry may comprise a temperature sensor. The temperature may be a thermistor that measures a body temperature of the patient. The temperature sensor may comprise any one of a thermistor, a resistance temperature detector (RTD), electrodermal activity (EDA) sensor, pyroelectric detector or a thermocouple. The temperature sensor may be flexible.

The temperature sensor may be a passive temperature sensor. The passive temperature sensor may be a thermistor which requires a current applied to measure the voltage. The temperature sensor may be a thermocouple that measures the temperature in dependence on the voltage between two different electrical conductors, e.g. two different metals.

The first and/or second sensor componentry may comprise a colour sensor. The colour sensor may be configured to detect a colour of the skin of the patient. The first and/or second sensor componentry may comprise any one of a PCO₂, SPO₂ or pulse oximeter sensor that measures the colour of skin. The first and/or second sensor componentry may detect one or both of respiratory rate and colour of the skin.

The first and/or second sensor componentry may comprise at least one film that changes colour in response to a change in a parameter of the patient. The first and/or second sensor componentry may comprise a colour sensor configured to sense the colour of the film. The film may be a thermochromic film that changes colour in response to a change of temperature of the patient. The film may, for example, change colour to reflect a detected CO₂ concentration in the patient's blood (PCO₂).

A PCO₂ sensor may measure the parameter precisely or approximately. The first and/or second sensor componentry may be configured to measure abrupt variations in PCO₂. An alarm may be triggered if the variation in the PCO₂ value is above or below a set threshold. The first and/or second sensor componentry measure the partial pressure of venous carbon dioxide (PvCO₂). This measurement may be taken remotely and/or wirelessly.

The first and/or second sensor componentry may comprise a motion sensor. The motion sensor may comprise a microelectromechanical system (MEMS) accelerometer. The motion sensor may detect any movement of the patient. The motion sensor may detect a breathing motion or breathing action of the patient

The first and/or second sensor componentry may comprise an electrocardiogram (ECG) sensor.

The first and/or second sensor componentry may comprise a pulse oximeter sensor. The pulse oximeter sensor may determine at least a saturation of peripheral oxygen (SPO₂) in the blood of the patient.

One or more electronic components for reflectance pulse oximetry may be disposed on the patient-facing side of the PCB. One or more signal processing components may be disposed on the fixation structure. The signal processing component(s) may be provided on one or both of the first side and second side of the body. For example, one or more signal processing components may be disposed on either side of the PCB Alternatively, signal processing may be performed externally to the patient interface, such as by a patient monitor, a respiratory therapy device, or other external device.

The first and/or second sensor componentry may comprise at least one light source. The light source may be a photodiode or LED. The first and/or second sensor componentry may comprise a photoplethysmography (PPG) sensor. The PPG sensor may utilise the light source and a photodetector. The PPG sensor may make an optical measurement of the arterial volume using the light source. The PPG sensor may be used to measure a blood flow in the patient.

The fixation structure may comprise an aperture. The first and/or second sensor componentry may be located in or adjacent to the aperture. The sensor may sense a parameter of the patient when the fixation structure is attached to the patient, for example, via the skin of the patient. The sensor adjacent to or in the aperture may be a colour sensor or pulse oximeter sensor.

In some configurations, the patient fixation structure may comprise a thinned region. Any one or more of the body, first extension, second extension, main region, patient attachment and/or fixing element may comprise a thinned region. The patient attachment may comprise a thinned region. For example, where the patient attachment comprises an adhesive, the layer of adhesive may be thinned in the thinned region. The first and/or second sensor componentry may be located adjacent to or on the thinned region(s).

A sensor of the first and/or second sensor componentry may be located adjacent to or on the thinned region(s). The sensor may sense a parameter of the patient through the thinned region(s) when the fixation structure is attached to the patient, for example, via the patient's skin. The sensor adjacent to or on the thinned region(s) may be a colour sensor or pulse oximeter sensor.

The sensor componentry may detect and/or retain usage information. For example, the sensor componentry may detect the duration of use of a patient interface device. The usage duration information may be provided to or transmitted to a user device, which indicate to a care giver that a device needs to be replaced. The sensor componentry may be part of a system that gives an alarm when a usage period has expired, or which informs the caregiver that the usage period has expired.

The sensor componentry may comprise a detector which determines when a patient interface is attached to the fixation structure. The sensor componentry may detect when a patient interface is removed from a fixation structure.

The sensor componentry may detect when the fixation structure is attached to the skin of a patient. The sensor componentry may detect when an element, part or all of the sensor componentry is removed from the skin of a patient.

According to an aspect of the present disclosure there is provided a fixation structure for securing a patient interface to a patient. The fixation structure comprises a body having a first side and a second side, the body being removably attachable to the patient. The fixation structure comprises a fixing element on the body, the fixing element configured to removably attach to the patient interface. The fixation structure comprises sensor componentry provided on or in one or more of the body, and fixing element or configured to be attached to one or more of the body and fixing element.

The fixation structure of this aspect may according to some configurations comprise any one or more features as disclosed herein in relation to any other aspect, embodiment or configuration or example of the present disclosure.

According to an aspect of the present disclosure there is provided a fixation structure for securing a patient interface to a patient. The fixation structure comprises a body having a first side and a second side. The fixation structure comprises a fixing element on the body. The fixation structure comprises sensor componentry provided on or in one or more of the body and fixing element or configured to be attached to one or more of the body and fixing element.

The fixation structure of this aspect may according to some configurations comprise any one or more features as disclosed herein in relation to any other aspect, embodiment, configuration or example of the present disclosure.

The fixation structure may comprise any feature as described herein in relation to a fixation structure, whether that feature is described individually or in combination with one or more other features.

According to an aspect of the present disclosure, there is provided a securement system for a patient interface for delivery of a breathable gas to a patient, the securement system comprising: the fixation structure according to any aspect, configuration or example disclosed herein; and a surface of a patient interface configured to removably attach to the fixation structure.

A further aspect of the present disclosure provides a patient interface for delivering a breathable gas to a patient, the patient interface comprising a securement system comprising: the fixation structure according to any aspect, configuration or example disclosed herein; and a surface of a patient interface configured to removably attach to the fixation structure.

The patient interface may further comprise an interface fixing element that is removably attachable to the fixing element of the fixation structure.

The interface fixing element may comprise one of a hook fastener and loop fastener; and the fixing element comprises the other of the hook fastener and loop fastener.

The patient interface may comprise any one of: a nasal cannula, face mask, nasal mask, oro-nasal mask, ET tube, or other breathable gas delivery structure.

The patient interface may comprise one or more prongs for delivery of gases to the patient. The patient interface may comprise a manifold for directing a breathable gas to each prong. The manifold may be fluidly connectable to a tube for delivering a breathable gas from a gas source.

The patient interface may comprise at least one wing or arm. The surface of the patient interface to which the fixation structure is removably attachable may be located on a patient facing side of the wing or arm. The interface fixing element may be provided on the wing or arm.

The patient interface may comprise any feature as described herein in relation to a patient interface, whether that feature be described individually or in combination with one or more other features.

According to an aspect of the present disclosure there is further provided a system for delivery of a breathable gas to a patient, comprising: the patient interface as provided above or as described herein; a gas source; and a tube fluidly connecting the patient interface to the gas source.

According to another aspect of the present disclosure, there is provided system for delivery of a breathable gas to a patient, comprising: a patient interface configured to communicate with an airway of the patient during use to deliver the breathable gas to the patient; and the fixation structure as provided by any aspect, configuration or example described herein, wherein the fixing element of the fixation structure is removably attachable to the patient interface, such that in use the patient interface is secured to the patient via the fixation structure.

The system of this aspect may comprise any one or more features as disclosed herein in relation to any other aspect, embodiment, configuration or example of the present disclosure.

According to another aspect of the present disclosure, there is provided system for delivery of a breathable gas to a patient, comprising: a patient interface; and the fixation structure as provided by any aspect, configuration or example described herein, wherein the fixing element of the fixation structure is removably attachable to the patient interface. In use the patient interface may be secured to the patient via the fixation structure.

The system of this aspect may comprise any one or more features as disclosed herein in relation to any other aspect, embodiment, configuration or example of the present disclosure.

According to another aspect of the present disclosure, there is provided a system for delivery of a breathable gas to a patient, comprising: a patient interface configured to communicate with an airway of the patient during use to deliver the breathable gas to the patient; and a pair of the fixation structures, each fixation structure being in accordance with any aspect, configuration or example described herein, wherein the fixing elements of each of the pair of fixation structures is removably attachable to the patient interface, such that in use a first fixation structure of the pair of fixation structures attaches the patient interface to the patient in one location and the second fixation structure of the pair of fixation structures attached the patient interface to the patient in a second location.

The system of this aspect may comprise any one or more features as disclosed herein in relation to any other aspect, embodiment, configuration or example of the present disclosure.

According to another aspect of the present disclosure, there is provided a system for delivery of a breathable gas to a patient, comprising: a patient interface; and a pair of the fixation structures, each fixation structure being in accordance with any aspect, configuration or example described herein, wherein the fixing elements of each of the pair of fixation structures is removably attachable to the patient interface. In use a first fixation structure of the pair of fixation structures may attach the patient interface to the patient in one location and the second fixation structure of the pair of fixation structures may attach the patient interface to the patient in a second location.

The system of this aspect may comprise any one or more features as disclosed herein in relation to any other aspect, embodiment, configuration or example of the present disclosure.

According to another aspect of the present disclosure, there is provided a system for delivery of a breathable gas to a patient, comprising: a patient interface configured to communicate with an airway of the patient during use to deliver the breathable gas to the patient; and a pair of fixation structures, each fixation structure comprising: a body having a first side and a second side, in use the first side being removably attachable to the patient; a fixing element on the second side of the body, the fixing element being removably attachable to the patient interface; and sensor componentry provided on or in the body, and/or fixing element or attachable to the body and/or fixing element, wherein the fixation structures in use attach the patient interface to the patient at two respective locations.

The system of this aspect may comprise any one or more features as disclosed herein in relation to any other aspect, embodiment, configuration or example of the present disclosure.

According to another aspect of the present disclosure, there is provided a system for delivery of a breathable gas to a patient, comprising: a patient interface; and a pair of fixation structures, each fixation structure comprising: a body having a first side and a second side; a fixing element on the second side of the body, the fixing element being removably attachable to the patient interface; and sensor componentry provided on or in the body, and/or fixing element or attachable to the body and/or fixing element. The fixation structures in use attach the patient interface to the patient at two respective locations.

The system of this aspect may comprise any one or more features as disclosed herein in relation to any other aspect, embodiment, configuration or example of the present disclosure.

According to another aspect of the present disclosure, there is provided a system for delivery of a breathable gas to a patient, comprising: a patient interface configured to communicate with an airway of the patient during use to deliver the breathable gas to the patient; and a fixation structure for securing the patient interface to the patient, the fixation structure comprising: a body comprising a main region, and a first extension and a second extension on either end of the main region, each of the main region, the first extension and the second extension having a first side and a second side, each of the first extension and the second extension being pivotally attached to and/or removable from the main region, wherein the first side of the main region is removably attachable to the patient; a first fixing element on the first extension, the first fixing element being removably attachable to the patient interface; a second fixing element on the second extension, the second fixing element being removably attachable to the patient interface; and at least one sensor componentry provided on or in one or more of the body, first fixing element and second fixing element or attachable to one or more of the body, first fixing element and second fixing element, wherein in use one of the first extension and the second extension pivots relative to the main region to attach to the second side of the main region, the main region attaching to the patient in a first location, and the other of the first extension and the second extension is removable from the main region and attachable to the patient in a second location.

The system of this aspect may comprise any one or more features as disclosed herein in relation to any other aspect, embodiment, configuration or example of the present disclosure.

According to another aspect of the present disclosure, there is provided a system for delivery of a breathable gas to a patient, comprising: a patient interface; and a fixation structure comprising: a body comprising a main region and a first extension and a second extension each removably attached to the main region, each of the main region, the first extension and the second extension having a first side and a second side, each of the first extension and the second extension being pivotally attached to and/or removable from the main region; a first fixing element on the first extension; a second fixing element on the second extension; and at least one sensor componentry provided on or in one or more of the body, first fixing element and second fixing element or attachable to one or more of the body, first fixing element and second fixing element.

In use, one of the first extension and the second extension may pivot relative to the main region to attach to the second side of the main region, the main region attaching to the patient in a first location. The other of the first extension and the second extension may be removed from the main region and attached to the patient in a second location. The system of this aspect may comprise any one or more features as disclosed herein in relation to any other aspect, configuration or example of the present disclosure.

The system is configured such that, in use, a feeding tube, for example a naso-gastric or oro-gastric feeding tube, may be secured on the patient at the pivot between the main region and the first extension. The system is configured such that, in use, a feeding tube may be secured on the patient at the pivot between the main region and the second extension.

The patient interface may comprise one or more of: a nasal cannula assembly, nasal prongs, face mask, nasal mask, oro-nasal mask, or other breathable gas delivery structure.

The or each fixing element may comprise a hook or loop fastener and the patient interface comprises at least one complimentary fastener, such that in use the hook or loop fastener attaches to the complimentary fastener.

According to another aspect of the present disclosure, there is provided a patient monitoring system, comprising: at least one patient interface for delivering a breathable gas to a patient; at least one fixation structure as provided by any aspect, configuration or example described herein, where each fixation structure is attachable to a patient interface; and a monitoring device that receives data sensed by the sensor componentry of each fixation structure.

The monitoring device may be configured to display the received data to the patient and/or a caregiver of the patient. The patient monitoring system of this aspect may comprise any one or more features as disclosed herein in relation to any other aspect, configuration or example of the present disclosure.

The patient interface may deliver the breathable gas to the patient from a respiratory therapy device. The monitoring device may be connected to or form part of the respiratory therapy device.

The monitoring device may be a mobile device, tablet, laptop or other computer device. The monitoring device may receive the data sensed by the sensor componentry wirelessly.

The patient monitoring system may comprise an alarm that is activated by the monitoring device. The alarm may be activated when the received data is above or below a threshold value. The alarm may be activated when the received data is outside a predetermined range.

### BRIEF DESCRIPTION OF DRAWINGS

The present disclosure will now be described with reference to the accompanying drawings. It is to be understood that the configurations disclosed are given by way of illustration only and the present disclosure is not limited by this illustration. In the drawings:

FIG. 1(a)-(e) shows a fixation structure with sensor componentry according to a first configuration;

FIG. 2 shows a top view of a fixation structure with sensor componentry according to a second configuration;

FIG. 3 shows a top view of a fixation structure with sensor componentry according to a third configuration;

FIG. 4 shows a cross sectional view of a fixation structure with sensor componentry according to a fourth configuration;

FIG. 5 shows a cross sectional view of a fixation structure with sensor componentry according to a fifth configuration; and

FIG. 6 shows a cross sectional view of a fixation structure with sensor componentry according to a sixth configuration;

FIG. 7 shows an example of a respiratory therapy system;

FIG. 8 shows a schematic diagram of a closed loop control system for use with a respiratory support apparatus;

FIG. 9 shows a front perspective view of a nasal cannula including two fixation structures in accordance with an aspect of this disclosure; and

FIG. 10 shows an exploded view of the nasal cannula of FIG. 9.

FIG. 11 shows a side schematic view of an example electrochemical cell with a reservoir.

FIG. 12 shows a side schematic view of another example electrochemical cell with multiple membranes.

FIG. 13 shows an exploded schematic view of a further example electrochemical cell with an outer layer.

### DETAILED DESCRIPTION

Patients suffering from various health conditions and diseases can benefit from respiratory support. As part of providing a patient with respiratory support, one or more physiological parameters and/or respiratory support system parameters can be measured and/or monitored by a sensor. The sensor may be configured to measure and/or monitor one or more of patient temperature, patient blood oxygen saturation (SpO₂), heart/pulse rate, breathing rate, carbon dioxide concentration in patient blood (PCO₂), skin colour, patient movement or patient interface usage duration. When a sensor, such as a patient sensor, is used as part of the respiratory support system, a user, is required to mount the patient sensor to the patient separately from attaching any respiratory apparatus such as a patient interface. This adds another task to that required to set up the respiratory support system. Additionally, a separate sensor can lead to problems such as incorrect mounting of the sensor leading to incorrect measurement/monitoring and/or the sensor falling off during use.

The user of the system may be for example a nurse, doctor, any other medical clinician or health care worker, or the patient themselves.

A patient interface/respiratory system which incorporates a patient sensor allows use of a sensor without increasing the workload of a user. This may be beneficial in a hospital setting, and may provide simplification of respiratory system and patient monitoring set up. This may also have benefits in a home setting, such as potentially simplifying set up process for a patient, who may need to perform the setup tasks themselves. Integrating a patient sensor into an aspect of the patient interface/respiratory system may also help to ensure correct orientation/placement of patient sensor.

A patient sensor in a fixation structure, also referred to as a dermal patch, which connects a patient interface to a patient as disclosed herein may be beneficial. For example, where a patient changes to a new therapy system and/or requires a different type of respiratory support, the dermal patch may remain constant. As the dermal patch may provide a connection point/fastener for the patient interface, it may allow switching between interfaces without any disruption of patient monitoring. In other words, elements of the respiratory therapy system may be changed while the dermal patch remains attached to the patient and still able to provide sensor data. Providing a sensor on, in or configured for attachment to, the fixation structure may also allow for the sensor to be quickly and easily replaced where necessary or to be removed entirely with ease by removing the fixation structure from the patient.

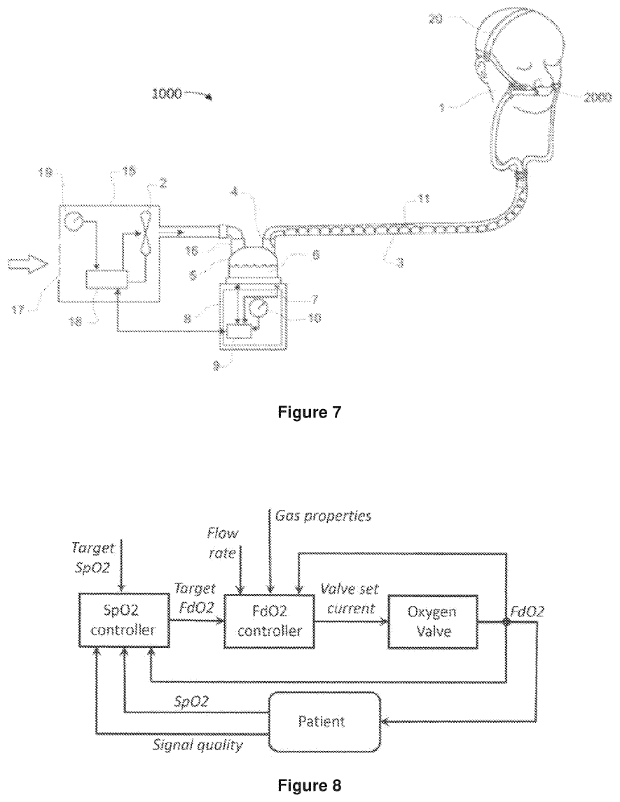

A schematic representation of an example respiratory therapy apparatus (or respiratory therapy system) is shown in FIG. 7.

The respiratory therapy system comprises a flow source or gas source for providing a gas such as air, oxygen, air blended with oxygen, or a mix of air and/or oxygen and one or more other gases. The system can have a connection for coupling to a flow or gas source. Depending on the configuration, as some components may be optional, the system can include a combination of components selected from: a flow/gas source; a humidifier for humidifying gas flow; conduit(s) e.g. dry line and/or heated breathing tube; and/or a patient interface.

The conduit may comprise an inspiratory conduit, an expiratory conduit, or gas inlet conduit.

A flow/gas source could be an in-wall supply, a tank and/or a flow source with a flow generator. The flow generator may have an optional air inlet and optional connection to an O₂ source, such as tank or O₂ generator. Flow generator can control flows delivered to the patient using one or more valves, or may comprise a blower, as shown in FIG. 7. The flow source can be one or a combination of: flow generator, O₂ source, and/or air source. The flow/gas source provides a flow of gas that can be delivered to a patient via an inspiratory conduit and patient interface. The flow source may provide a base gas flow rate of between about 0.5 LPM and about 375 LPM or any suitable sub-range within that range.

The blower 15 may be provided with a variable speed pump or fan 2 that draws air or other gases through a blower inlet 17. The speed of the variable speed pump or fan 2 may be controlled by a further control means or electronic controller 18 (or alternatively the function of the electronic controller 18 could be carried out by the controller 9) in response to inputs from the controller 9 and a user-set predetermined required value (preset value) of pressure and/or fan speed via one or more input devices 19.

The patient interface may be an unsealed (non-sealing) interface, for example when used in high flow therapy, such as a non-sealing nasal cannula. Alternatively, the patient interface may be a sealed or sealing interface, for example when used in CPAP, such as a nasal mask, full face mask, nasal cannula or nasal pillows. The patient interface may also include tracheostomy interface or endotracheal tube (ET tube).

A humidifier may optionally be provided between the flow source and the patient to provide humidification of the delivered gas. A humidifier is configured to combine or introduce humidity with or into the gases flow. Various humidifier configurations may be employed. The humidifier may comprise a humidification chamber. The humidification chamber may be removable, for example, may be partially or entirely removable or disconnected from the flow path and apparatus.

The humidification chamber may comprise a gases inlet and a gases outlet to enable connection into the gases flow path of the apparatus/system. For example, flow of gases from flow generator is received into the humidification chamber via its gases inlet and exits the chamber via the gases outlet, after being heated and/or humidified. The humidification chamber may contain a volume of liquid, typically water or similar. In operation, the liquid in the humidification chamber is controllably heated by one or more heaters or heating elements associated with the chamber to generate water vapour or steam to increase the humidity of the gases flowing through the chamber.

In one configuration, the humidifier is a pass-over humidifier.

In one configuration, the humidifier may comprise a heater plate, for example associated or within a humidification bay that the chamber sits on for heating. The chamber may be provided with a heat transfer surface, such as a metal insert, plate or similar, in the base or other surface of the chamber that interfaces or engages with the heater plate of the humidifier.

The humidification chamber may be any suitable shape and/or size. The location, number, size, and/or shape of the gases inlet and gases outlet of the chamber may be varied as required. In one configuration, the humidification chamber may have a base surface, one or more side walls extending up from the base surface, and an upper or top surface. In one configuration, the gases inlet and gases outlet may be position on the same side of the chamber. In another configuration, the gases inlet and gases outlet may be on different surfaces of the chamber, such as on opposite sides or locations, or other different locations

An inspiratory conduit 3 may be coupled to a gases flow outlet of the respiratory therapy system and may be coupled to the patient interface. A heating element 11 may be provided within the inspiratory conduit 3 to help prevent condensation of the humidified gases within the conduit 3.

Gas flow, of one or more gases, may be generated by the flow generator and may be humidified before being delivered to the patient via inspiratory conduit and patient interface. A controller may control the flow generator to generate a gas flow of desired flow rate, and/or one or more valves to control mixing of air, O₂ or other breathable gas. The controller may control a heating element in or associated with the humidification chamber, where present, to heat gases to a desired temperature in order to achieve a desired level of temperature and/or humidity for delivery of the gases to the patient. The inspiratory conduit can have a heating element, such as a heater wire, to heat gases flow passing through to the patient. The heating element can also be under control of controller.

With further reference to FIG. 7, the respiratory therapy system 1000 may provide a patient 1 with humidified gases through a patient interface 2000. The patient interface 2000 can supply humidified and pressurised gases to the patient 1 during respiratory therapy. In the example shown in FIG. 7, the patient interface 2000 is a nasal cannula that is supplied with gases from a gas supply means such as a respiratory therapy device 1000. The patient interface 2000 shown in FIG. 7 includes headgear 20 to support and retain the patient interface on the patient, which in this case is on the patient's face. The headgear 20 may be optional. In accordance with the present disclosure, at least one fixation structure may be used to attach the patient interface 2000 to the patient 1.

The nasal cannula assembly may be connected to a humidified gases transportation pathway or inspiratory conduit 3. The inspiratory conduit 3 may be connected to a humidifier 8, including a humidification chamber 5, that is supplied with gases from a gas supply means, blower 15 or other appropriate gases supply means. The gas or gases can be supplied from a source that is external to and/or separate from the respiratory therapy system 1000, or from a source that is internal to and/or integrated with the respiratory therapy system 1000.

With continued reference to FIG. 7, an inspiratory conduit 3 is connected to an outlet 4 of the humidification chamber 5 which contains a volume of water 6. Optionally, the system may have an intermediate conduit between the outlet 4 of the humidification chamber 5 and the inspiratory conduit 3. The inspiratory conduit 3 fluidly connects to the outlet 4 of the humidification chamber 5 to receive the humidified gases and to transport them to the patient interface. The humidification chamber 5 may be formed from a plastics material and may have the heat transfer surface referred to above (for example an aluminium base) which may be in direct contact with a heater plate 7 of the humidifier 8. The humidifier 8 may be provided with a control mechanism or electronic controller 9, such as a microprocessor based controller, executing computer software commands stored in associated memory. Gases flowing through the inspiratory conduit 3 are passed to the patient 1 by way of the patient interface 2000.

The controller 9 may receive an input from an input device 10, through which a user may set a predetermined required value (preset value) of humidity or temperature of the gas supplied to the patient. In response to the user-set humidity or temperature value input and other possible inputs such as internal sensors that sense gas flow or temperature, or other parameters, the controller 9 determines when (or to what level) to energize the heater plate 7 to heat the water 6 within the humidification chamber 5. As the volume of water 6 within the humidification chamber 5 is heated, water vapor begins to fill the volume of the humidification chamber 5 and is passed out of the outlet 4 with the flow of gas (for example air) provided by the blower 15 which enters the humidification chamber 5 through an inlet 16. It is possible to determine a relationship between the humidity of the gases in the humidification chamber 5 and the temperature of the heater plate 7. Accordingly, it is possible to utilize the temperature of the heater plate 7 in an algorithm or a look-up table to determine the humidity of the gases.

The patient interface may include a nasal cannula, a nasal mask, an oronasal or full face mask, an endotracheal (ET) tube, or any suitable structure configured to deliver a breathable gas to a patient/user. The patient interface may include at least one interface attachment element that releasably affixes the patient interface to at least one fixation structure on the patient. The interface attachment element(s) may maintain the patient interface in a desired alignment with the patient's face such that respiratory therapy is delivered effectively.

According to an example, nasal prongs on a nasal cannula may be comfortably and non-sealingly positioned in the patient's nares when respiratory therapy, such as NHF therapy, is being delivered, and may assist to prevent dislodgement of the prongs. Maintaining the relative positioning of the patient interface and patient's face is also used for sealed system respiratory therapy, such as CPAP and ET tubes, where the patient interface needs to be positioned correctly and stabilised.

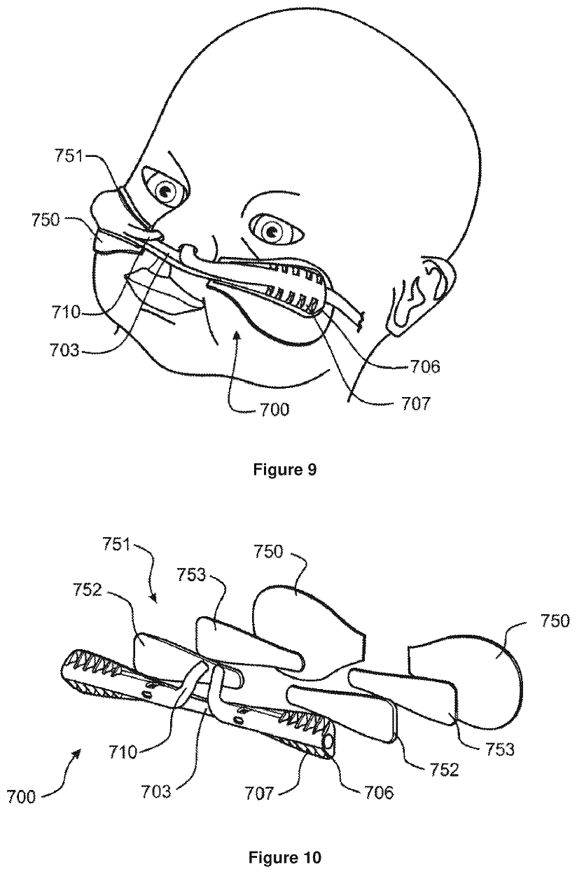

FIGS. 9 and 10 show an example embodiment of a patient interface 700, which in this example, is a nasal cannula. In this example, the nasal cannula broadly comprises body, a gases inlet conduit, connector and a securement assembly. The securement assembly enables a user to place and maintain the nasal cannula in the correct operational position. The gases inlet conduit forms a fluid or gases connection between the outlet end of the inspiratory conduit and the nasal cannula to allow fluids or gases to flow between the inspiratory conduit and nasal cannula. The connector may be, in use, connected to and in fluid communication with the gases inlet conduit.

The body of the patient interface 700 may include a pair of nasal prongs 710 extending from a base portion of the body. Gases flow may pass through the body to the nasal prongs 710 and is delivered to the patient. In some configurations, the nasal prongs 710 may be in fluid communication with each other, such as via a manifold 703. The manifold 703 is in turn in fluid communication with the gases inlet conduit. In other configurations, the nasal prongs may each have an independent flow path. Each prong may have separate delivery tubing. The delivery tubing may be fluidly connected at the connector end of the interface.

With continued reference to FIGS. 9 and 10, the patient interface comprises a pair of wings 707 located at opposed ends 706 of the patient interface. respective tube for delivering a breathable gas. Alternatively, each wing 707 may comprise a conduit that fluidly connects a respective prong to the tube for delivering a breathable gas and the patient interface comprises a manifold to deliver a gas from the tube to each respective prong.

The patient interface 700 is attachable to a fixation structure as described herein, for example as shown in FIGS. 9 and 10. The patient interface 700 may include a surface on the patient facing side of each wing 707 to which the interface attachment element 752 is attachable. The interface attachment element 752 has a patient side and an interface side. The interface side of the interface attachment element 752 is attachable or affixed to the patient interface 700. The interface attachment element 752 may be integrated with or suitably adhered to the patient interface 700.

The fixation structure includes a body 750 and fixing element 753. The fixation structure has a patient side that faces the patient's skin and an interface side that faces the patient interface 700. The interface side of the fixation structure is provided with a fixing element 753. The fixing element 753 is the first part of a two-part releasable securement assembly 751. A second part of the two-part releasable securement assembly 751 is the interface attachment element 752. The patient facing side of the interface attachment element 752 is attachable to the interface facing side of the fixing element 753. The releasable securement assembly 751 acts between, and releasably connects, each of the pair of fixation structures that are affixed to the patient with the patient interface 700 respectively.

The body 750 of the fixation structure may be adhered or otherwise attached to the patient's skin. The patient side of the fixation structure body 750 may be attached to the skin of a patient by a dermatologically sensitive adhesive. The adhesive may include any one or more of: a hydrocolloid-based adhesive material; a zinc oxide-based adhesive material; a silicone-based adhesive material; a polyurethane; and/or a hydrogel-based adhesive material.

The two-part releasable securement system 751 may comprise complementary fastening elements. For example, the two-part releasable securement system may comprise a mechanical fastener, such as a hook and loop material (such as Velcro™), a magnet or an array of magnets disposed respectively on each of the fixation structure(s) and patient interface 400 having the poles suitably arranged, an adhesive arrangement that may be activated when the two parts are brought together, or any other suitable releasable coupling. The interface side of the fixation structure 750 may have one of a hook or a loop material, and the patient side of the interface attachment element 752 may have the other of the hook or loop material, such that the fixation structure 750 and interface attachment element 752 are releasably attachable to each other.

One or more interface attachment elements may couple to fixation structures that are attached to the patient, for example, on the face. In other words, the fixation structure(s) may already be attached to the patient before the patient interface is attached. Alternatively, the patient interface may be attached to the fixation structure and then the fixation structure with patient interface attached may be fixed to the patient.

According to the present disclosure, the fixation structure(s) may comprise a patient-facing region that removably attaches the fixation structure to the patient. The fixation structure may also comprise an interface-facing region that is configured for attachment to the interface attachment element on the patient interface. For example, the interface-facing region may comprise the fixing element. According to other configurations, the patient interface does not comprise an interface attachment element and the fixation structure's fixing element(s) removably attach directly to a region of the patient interface. Similarly, configurations are possible where the fixation structure does not comprise a fixing element and the interface attachment element(s) on the patient interface attach directly to an interface facing region of the fixation structure. For example, an adhesive with suitable properties as discussed herein may be used in place of one or both of the fixing element and interface attachment element.

More detail on/examples of patient interfaces and/or fixation structures and their means of attachment to one another and the patient are provided in the following PCT publications by the present applicant: WO2012/053910A1, WO2015/057083, WO2016/159783A1 and WO2016/148585A1, the contents of each of which are incorporated herein by reference. The present disclosure should be interpreted broadly and it is understood that the present disclosure should not be restricted by reference to any specific embodiments disclosed herein or in any of these PCT publications. The concepts discussed herein are intended to be applicable to many types of patient interfaces and fixations structures equally, whether specifically disclosed herein or not.

As a non-limiting example, the following description refers to some features of FIG. 4 for assistance in understanding of the physical structure of the fixation structure. The features described are equally applicable to the examples shown in other Figures or any configuration or example of the present disclosure more broadly.

The patient-facing region of the fixation structure 400 may be on a first side 403 of the fixation structure 400. The interface facing region of the fixation structure may be on a second side 402 of the fixation structure 400. The second side 402 may be opposed to the first side 403 of the fixation structure 400.

The patient-facing region of the fixation structure 400 may be constructed at least in part from a material that can be removably attached to the patient. The patient-facing region of the fixation structure may include a material 410 that can be removably attached to the patient. The material 410 may be a dermatologically sensitive adhesive material. The material 410 may be an adhesive layer, which may be formed from one or more layers of adhesive material. Hereafter the material 410 is referred to as adhesive layer 410, however it is understood that this reference to adhesive layer could be any material that is suitable for attachment to the patient. The adhesive layer 410 may include any one or more of: a hydrocolloid-based adhesive material; a zinc oxide-based adhesive material; a silicone-based adhesive material; a polyurethane; and/or a hydrogel-based adhesive material. The adhesive layer 410 may be flexible for conformity to the contours of a patient. A removably attached protective backing sheet may be placed over the adhesive layer 410 prior to attachment of the adhesive layer 410 to the patient.

The fixation structure comprises a body 401. The body 401 may comprise a substrate. The body 401 of the fixation structure 400 may comprise a substrate material. The body 401 may be formed from one or a plurality of layers. The adhesive layer 410 that removably attaches the fixation structure 400 to the patient may be applied to the body 401, for example to a first side 403 of the body 401. The patient side 403 of the body 401 may be adhesively attachable to the skin of a user, and may be provided with a removably attached protective backing sheet. The body 401 may include a polymer material, for example, a polymer resin, plastic and/or composite. For example, the body 401 may comprise a urethane-based material or an imide-based polymer. An interface-facing side, or second side 402, of the body 401 may comprise the interface-facing region of the fixation structure 400 that attaches to the patient interface.

The adhesive layer 410 that removably attaches the fixation structure 400 to the patient may be breathable and/or moisture permeable. The body 401 of the fixation structure 400 may be at least partially breathable and/or moisture permeable. The fixation structure 400 may be at least partially breathable, in other words one or more layer (e.g. adhesive material, body and fixing element) of the fixation structure 400 may be breathable.

The fixation structure 400 as a whole may have a MVTR >0 g/m²/24 h. The MVTR of the fixation structure 400 may be at least about 100, 200, 400, 1000, 1500, 2000, 5000 or 10,000g/m²/24 h.

The adhesive layer 410 that removably attaches the fixation structure 400 to the patient may be flexible so as to conform to the contours of the patient, for example, the patient's face. Similarly, the body 401 of the fixation structure 400 may be flexible so as to conform to the contours of the patient. The fixation structure 400 as a whole may be flexible and/or configured to conform to the contours of a patient.

The fixation structure 400 comprises at least one sensor. The fixation structure 400 comprises sensor componentry 450. The sensor componentry 450 may comprise a PCB. The sensor componentry 450 may be located on the first side 403 and/or second side 402 of the body 401. The sensor componentry 450 may be positioned on the body 401. The sensor componentry 450 may integrally formed with the body 401. The sensor componentry 450 may be located on or within the adhesive layer 410. The fixation structure may be located on or within the fixing element 421.

Traces, or tracks, of the PCB may be formed on the body 401, for example, a conductive ink may be screen-printed onto the body 401 to form the traces/tracks. The PCB may form the electrical connections of the sensor componentry 450 that is provided on the body 401 of the fixation structure 400.

The sensor componentry may include at least one electronic component. The PCB may include one or more electronic components. The electronic component(s) may be selected from any one or more of resistor(s), capacitor(s), actuator(s), sensor(s), integrated circuit(s), coil(s), diode(s), transistor(s), and/or inductor(s). The electronic component(s) may be surface mounted onto the PCB or may be screen printed onto the body 401 of the fixation structure 400. Surface mount technology may be used together with an encapsulation material or coating resulting in a circuit embedded within a waterproof enclosure. The sensor componentry 450 is used to take a reading of one or more parameters when the fixation structure 400 is in use. The fixation structure 400 may be in use when attached to the skin of a patient and/or when attached to a patient interface.

In some embodiments, the adhesive layer 410 may be applied to the patient side 403 of the substrate after the sensor componentry 450 has been formed, such that the sensor componentry 450 may be at least partly embedded in the adhesive layer 410.

In some configurations the sensor componentry 450 may be electrically isolated from the patient and/or external elements and/or other components. To aid in electrically isolating the sensor componentry 450, the adhesive material 410 may be non-conductive. In other words, the adhesive layer 410 may encapsulate and/or insulate the sensor componentry.

The sensor componentry 450 may be waterproofed in some manner to avoid unwanted conductivity arising from exposure to moisture. In some configurations, where moisture permeable adhesives are utilised, it may be desirable to cover the sensor componentry 450 with a waterproof housing. For example, a moisture-impermeable layer may be placed between the PCB and/or the sensor componentry 450 and the adhesive layer 410. The waterproof housing may be a conformal coating that may be sprayed onto the PCB and/or sensor componentry 450, such as through vapour deposition.

In some configurations, the body 401 of the fixation structure 400 may comprise a moisture-permeable substrate material. In such configurations, where the sensor componentry 450 is to be isolated from exposure to a moisture, the sensor componentry 450 may be manufactured separately. The sensor componentry 450 may be placed or otherwise positioned inside a waterproof housing or encapsulated in a suitable material. For example, sensor componentry 450 may be overmolded with plastic material on both sides to form a waterproof housing and encapsulate the sensor componentry 450. The encapsulated sensor componentry 450 may, for example, be positioned between the adhesive layer 410 and the body 401. While the sensor componentry 450 may be waterproof, in some configurations, surrounding space outside of the sensor componentry 450 and space between components may comprise a non-waterproof material to assist in maintaining breathable and/or moisture permeable properties of the fixation structure 400. For example, the fixation structure 400 may be at least partially breathable and/or moisture permeable for improved comfort to the individual to whom the fixation structure 400 is attached.

In some configurations, the sensor componentry 450 may comprise a multi-layer arrangement. For example, the sensor componentry 450 may comprise a multi-layer PCB. The multiple-layer PCB may be located in, on, or attached to the fixation structure 400. Consecutive substrate layers can be attached to a previous adhesive layer. Some types of circuits may require multiple layers to function. Hence, the type of circuitry used may define how many layers are required. For example, the body 401 of the fixation structure 400 may comprise a plurality of substrate layers that are laid out one on top of the other. A multi-layer circuit for the sensor componentry 450 can, for example, be applied or screen printed directly onto a plurality of overlaid substrate layers to create the multi-layer arrangement.

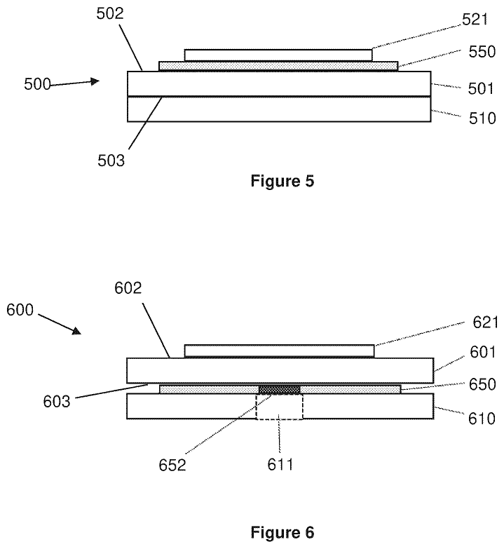

As described previously but with reference to FIG. 5 here, the fixation structure 500 may comprise an interface-facing region on the interface-facing side 502 of the body 501. The interface facing region may comprise any manner of fixing element 521 that may attach to the patient interface to removably couple the patient interface to the patient. For example, as previously discussed, a hook-and-loop material may be used.

In some configurations, sensor componentry 550 may be sized such that it is not suitable to be placed on the patient facing side 502 of the body 501. For example, the sensor componentry 550 may be thicker than the body 501 or adhesive material layer 510. The sensor componentry 550 may be placed on the interface-facing side 502 of the body 501.

In other configurations, there may be no specific substrate or the fixing element 521 may comprise the body 501. The body 501 may comprise the fixing element 521, such that the fixing element 521 may be attached directly to the skin of the patient via adhesive layer 510. In such configurations, the sensor componentry 550 may be applied to or located on or in the fixing element 521 of the fixation structure 500.

In some configurations, the hook-and-loop material of the fixing element 521 may have one or more holes. One or more components may be located in the hole(s) and/or nested inside the hook-and-loop material. In this way, any added height of the sensor componentry 550 may extend beyond the body 501 but not necessarily beyond an extent of the hook-and-loop material.

### Wireless Communication

The use of wireless communication to transmit sensor data may be useful since it can reduce the number of wires or cables used around or extending from the patient. In other words, the use of wireless communication may avoid ‘clutter’ around a patient. Where the patient is an infant, fewer wires/cables may improve physical interactions between parents and their infant, which can lead to improved outcomes for infant patients.

The use of wireless communications may also be beneficial for improved portability, such as for use in an ambulance, in combination with a mobile device or for ambulatory patients. Reducing or eliminating wired connections on a patient may also make transport between ambulance and hospital, Intensive Care Unit (ICU), Neonatal Intensive Care Unit (NICU) or between wards within a hospital an easier process. The use of wireless sensors may also assist in easing transition between respiratory therapy or when changing between patient interfaces to make the transition more convenient.

Data retrieved from the sensor(s) of the sensor componentry can be communicated to a secondary device or receiving station according to a wireless communications protocol. Additionally or alternatively the sensor componentry may receive information from an external source wirelessly via a wireless protocol. Any suitable method of wireless communications could be used to transmit or receive data. The chosen method of wireless communication may dictate the features of the sensor componentry or any wireless architecture that are necessary to permit implementation of that wireless method.

In some configurations, one or more inductive coils may be used to perform wireless communications between the sensor componentry and another device. An inductive coil can, for example, be imprinted on the substrate or adhesive. The inductive coil may be proximal to the sensor componentry. The inductive coil may be an integral component of the sensor componentry. The inductive coil(s) may form at least part of a wireless module. The wireless module may be on, in or connected to the fixation structure to transmits data from the sensor componentry and may be separate from the sensor componentry. The wireless module may form part of the sensor componentry.

The wireless module may comprise an antenna. The antenna may be used to send information acquired from the sensor(s) of the sensor componentry to an external device. The antenna may comprise, for example, at least one coil. The antenna may be used to receive information.

In some configurations, an antenna may be controlled to send out data at periodic intervals without any prompt from an external device. For example, the antenna may send out data at intervals of: every 5 seconds, every 10 seconds, every 20 seconds, every 30 seconds, every minute, every 2 minutes, every 5 minutes, every 10 minutes, every 15 minutes, every 20 minutes, every 30 minutes, every 40 minutes, every 45 minutes, every 50 minutes, every hour, every 2 hours, or as often as is desired for the specific sensor information to be sent out. Some sensor data may need to be sensed and/or transmitted on a more regular occurrence than other types of sensor information.

The interval of transmission may be selected for any specific data as desired. For example, patient heartbeat data may be sensed on a regular or continuous basis. Temperature data may be sensed intermittently. Pulse, SPO₂, colour and/or PCO₂ sensor data may be sensed continuously or for a continuous period of time separated by specified intervals of time. The transmission of sensor data may be on the same interval at which the data is sensed. Alternatively, the transmission of sensor data may be at a different interval to that at which the data is sensed. The data may be transmitted when the sensor data is within a specified range, above or below a threshold value and/or within a predetermined period of time following the previous data transmission. An external device may make a wireless request for data that prompts the wireless module/coil to transmit the requested data. For example, when a caregiver is performing an inspection, they may utilise their device to request up to date information on parameters of the patient they are inspecting.

Additionally or alternatively, one or more coils may be used as part of a wireless power transfer arrangement/system to provide power to one or more electronic components of the fixation structure, such as the sensor componentry. This is discussed further below.

The size of each loop and number of turns of each coil can be selected based on various power and shape constraints. The inductance of a coil is related to the number of turns it has, and the inductance value is a parameter used in the design of wireless power/communications systems. The number of coils may also be selected based on size and power requirements. The use of a plurality of coils may enable coils to be positioned in a plurality of different locations and/or orientations, which may result in less dependence on the location/orientation of a single coil. For example, the size of an inductive coil used is restricted by the size of the fixation structure to which it is attached, printed or otherwise connected. The inductive coil may be sized as large as possible within the physical constraints of the fixation structure body, adhesive or fixing element, depending on where the inductive coil is positioned on the fixation structure.

The positioning of the coils both on the fixation structure and relative to the skin of the patient when the fixation structure is attached to the skin are important considerations. Energy transfer through a coil may be most efficient when the coil is perpendicular to the direction of a magnetic field. The position and orientation of a receiving coil, relative to the transmitting coil, may affect the degree of magnetic coupling and therefore how much power is successfully transferred between the coils. Poorer coupling means the induced voltage/current in the receiving coil will be lessened. This may lead to a result that a smaller proportion of the energy input to generate a magnetic field on the transmitting side can be used by the receiving coil. Thus, the wrong orientation may result in a system that is less efficient. The patient could potentially move around or be positioned in a variety of positions. The magnetic field may have a constant direction.

In some configurations, two fixation structures may be used on the same patient. For example, one fixation structure may be provided on each side of the patients face. The patient interface may have connection regions on either side of the patient interface that attach to the fixation structures on the patient, for example, each side of the patient's face. In configurations where two fixation structures are used, one on each side of the face, or where a single fixation structure is split and used on either side of the patient's face, a respective coil may be included on the structure on each side of the face. In this case, at least one coil should be correctly oriented in relation to the magnetic field at any one time.

The coils on the structures on either side of the patient's face may be oriented such that a plane of each coil is angled relative to that of the other. For example, coils may be oriented in planes that are approximately 90 degrees to each other, or their planes may be substantially perpendicularly to one another. In other examples, there may be a non-perpendicular angle between the planes of each coil. In this manner, the patient should not be able to orient themselves in a manner that makes the axes of both coils substantially parallel to rather than substantially perpendicular to the applied magnetic field. The coils may transmit information or power more efficiently when perpendicular to the applied field. Having a plurality of coils in different orientations may increase the likelihood of having at least one coil oriented substantially perpendicularly to the applied field.

The coils may additionally or alternatively respond to different frequencies. The coil(s) may be tuned during the manufacturing or design process to permit transmission and/or receipt of information or power at a particular frequency. Some frequencies may be less affected by coil orientation than other frequencies. There may be a trade-off between the dependence on coil orientation and other factors such as a limited bandwidth or higher power requirement, etc. Therefore, there may not be one operational frequency that works most appropriately for all situations.

An optimised operational frequency may be determined for a particular patient setup that is used. If the fixation structure is intended to communicate with more than one piece of hardware at a time, this may be done by transmitting and/or receiving at different frequencies depending on the purpose, e.g. data or power, and/or the equipment with which it is communicating.

A receiver can couple to an antenna of the fixation structure to receive data and/or signals transmitted by the antenna. The receiver may be placed in proximity to the patient. For example, a receiver may located in any of: under the patient, in or on a bed on which the patient is located, in the respiratory therapy device, on a pole, in or on a table adjacent to the patient, in a portable device such as a mobile phone or handheld wand (small device that can energize and receive data from a passive sensor), in or on a display screen or on a cap on the patient's head.

Wireless communications may be used that utilise a suitable wireless communications protocol. For example, radio waves such as ultra-high frequency (UHF) radio waves may be used. Examples of wireless communications protocols that may be used to transmit or receive data, that may be used for transmitting data from sensor componentry or receive data at the sensor componentry, include: Bluetooth, Wi-Fi, RFID (radio frequency identification), BLE (Bluetooth low energy) or ANT+ (an open access multicast wireless sensor network technology).

The wireless communication may, for example, utilise one or more of the following networks: BAN (body area node/network), WBAN (wireless body area node/network), WLAN (wireless local area network) and/or WPAN (wireless personal area network). As examples, Bluetooth or Wi-Fi may be used when the sensors are powered independently or do not require power. For example, the fixation structure(s) may utilise a system that uses inductors to power the sensor componentry(s), but data from the sensors may be sent over Bluetooth, Wi-Fi or another wireless protocol. RFID is a form of wireless communication using electromagnetic and antenna coupling within the RF band. RFID generally uses a reader to interrogate a tag that contains a radio receiver and transmitter. RFID tags may be any of active (battery equipped), battery assisted or semi-passive (having a small embedded battery or capacitor that assists the circuit when interrogated) or passive (requiring no battery). The use of a semi passive power source may provide a compromise between purely passive circuits that have no battery and active circuits which contain a battery that are used continuously.

According to some situations, there may be a plurality of respiratory therapy devices and/or systems in a single hospital room or on a patient ward. There may be a plurality of patients in the same room or ward. Each patient may have a separate respiratory therapy system with their own patient interface and fixation structure(s). The sensor componentry(s) on a particular patient may be paired wirelessly with a respiratory therapy device supplying or delivering breathable gas to that patient, such that the sensor componentry only supplies information to the desired device. Alternatively or in addition, a communication device, such as a USB stick or dongle, may be connected to the particular therapy device to which the fixation structure is to be paired. The fixation structure may transmit data to this communication device which then communicates the data directly to the therapy device. This configuration eliminates the need to pair the fixation structure to a specific therapy device. Rather, it is paired to the communication device that is plugged in to the therapy device. The communication device can then be removed and plugged into another device or system, as necessary, such as when the patient is being moved or where a different device is required to access the data from the sensor componentry(s) on the fixation structure(s) on a given patient. The communication device may be able to plug into other patient therapy or monitoring systems to communicate the patient information to those systems/devices. Examples of such systems/devices include: ventilators, bedside monitors, monitoring systems, mobile devices such as tablets or mobile telephones, or any other system that may be found in a hospital ward, ICU or NICU.

In some configurations, data from sensors of the sensor componentry(s) may be transmitted to devices, for example, mobile phones, tablets or a wand using near field communication (NFC). NFC may be suitable for example, for a passive sensor device. NFC may be used to transmit identification information that requires very small amounts of data, for example, such as patient identifiers. NFC is range limited compared to other wireless methods such as Bluetooth or Wi-Fi, so it may be advantageous for a receiving device using NFC to be portable and positionable close to the fixation structure(s). As a healthcare provider may frequently be close to a patient to take a reading or monitor a patient, a close proximity receiving device using NFC is unlikely to require additional visits to the patient.

The receiving device that receives data from the sensor componentry may communicate received data to the respiratory therapy device. The receiving device(s) may communicate wirelessly or over a wired connection with the therapy device.

A mobile application may be used by the receiving device to log, track and share information over an independent network. In this example, the patient could be on any therapy device, as all data from the fixation structures may be managed separately and may not require compatibility with the therapy device.

### Power Supply

The power requirements of the sensor componentry may depend at least in part on the sensor and/or communication architecture and nature and number of components used. Some sensors may not require a large amount of power, such as a sensor that is intermittently active for a given period. For example, a temperature sensor may only be active for less than a second, such as around 100 ms, whereas an SPO₂ sensor may need to be active for about 30 seconds to obtain a suitable measurement. Such sensors may use a higher current at a shorter duration than other sensors. Some sensors, such as LEDs, or photodiode or phototransistor pairs for pulse oximetry, that may run continuously may require comparatively larger amounts of energy. In one example, an LED may require about 10-20 mA of current. A traditional blood oximeter may, for example, consume about 20-60 mW of power during continuous operation, with the LED being the main use of power. The power usage can be reduced by reducing the period of time the LED is active (i.e. increasing the time the LED is switched off), such that measurements are taken intermittently rather than continuously.

As discussed above, the sensor componentry circuitry in the fixation structure may be powered wirelessly. The circuitry may comprise a receiving coil that receives power from an emitting coil. This may, for example, utilise closely coupled inductive power transfer (IPT), where the emitting coil is in close proximity to a coil or coils in the fixation structure during use to facilitate coupling via the magnetic field generated by the emitting coil. In one example, the emitting coil may be within 10 cm of the fixation structure.

Alternatively or additionally, one or more sensor componentry can be battery powered. A dry cell battery may be used. The battery may be selected from any one of: a zinc-carbon cell, an alkaline cell, a lithium cell, a mercury cell, a silver-oxide cell, a nickel-cadmium cell, a lithium-ion cell or nickel-metal hydride cell. The battery may be a thin-film battery. The battery may be incorporated in the fixation structure. Power may be required at regular, and/or infrequent intervals, for example, every 30 s, to take and or transmit a reading. In this case overall a very low amount of power may be required, which would make a thin film battery suitable.

Surface mount batteries and supercapacitors may also be used. These may be utilised, for example, where higher levels of power are required.

Alternatively or additionally, the circuitry can harvest energy from external sources. The harvested energy can be used to directly power the circuit, or can be stored in a battery on the fixation structure or connected to the fixation structure which then powers the sensor componentry(s). For example, energy may be harvested from solar, piezoelectric, electromagnetic or thermal sources. In some configurations, a thermoelectric battery may be used. The thermoelectric battery may, for example, utilise the Peltier effect to convert heat energy from the body of the patient or a suitable heated surface (such as a dark coloured surface that absorbs radiant light energy from the sun or a heat lamp) to electrical energy.

The battery may be configured to supply power once the fixation structure is attached to the patient's skin. In some configurations, a switch or button could be used to activate the battery to power the sensor componentry(s). Alternatively or additionally, when the fixation structure is placed on the patient, the patient's skin may complete a circuit to activate the battery.

Some of the elements of the sensor componentry(s) may be passive or semi-passive whereby the electronics do not directly require a constant or consistent power source. Passive components may have the advantage of being physically small and easy to integrate into the fixation structure without comprising of many components.

The use of passive components may be limited to instant measurements, as the energy for data transfer and/or sensing comes from the data receiving device, such as a mobile phone, upon the request for data from the device.

In some configurations, data communication and wireless power may be provided through the same method or architecture. In other configurations, they may be provided through different methods or architecture. In some configurations, for example, data communication may be wireless while power may be supplied through a battery or batteries or a wired power supply. Alternatively or additionally, power may be supplied wirelessly while data communication is provided through a wired connection.

The fixation structure may incorporate one or more low-power sensors that can measure and transmit patient parameters or metrics wirelessly.

Alternatively or additionally, the sensor componentry and/or circuitry thereof may receive power from an electrochemical cell.

Electrochemical cells may be configured to convert chemical energy to electrical energy through electrochemical reactions, typically, redox (reduction-oxidation) reactions.

Electrochemical cells may typically comprise of electrodes being a cathode and an anode that in the presence of an electrolyte carrying ions facilitate redox reactions. During operation, the anode undergoes oxidation, releasing electrons to the cathode, which undergoes reduction. This process generates an electrical current that may be used to power a device or stored in a battery.

In some examples, the electrochemical cell may comprise a fuel cell, a biofuel cell and/or an enzymatic biofuel cell.

In some examples, the electrochemical cell may be configured to generate electrical energy in the presence of a fuel, also known as a substrate, and at least one compound or composition. The at least one compound or composition may comprise one or more of an electrolyte and/or catalyst.

Electrochemical cells may generally comprise electrodes such as at least one anode and at least one cathode, where oxidation of the fuel may occur at the anode and reduction of the oxidant may occur at the cathode to thereby generate electrical energy.

The electrodes may be made of conventional materials, e.g. gold, platinum or carbon, and/or biocompatible conducting polymers, nanoparticles, nanofibers, nanotubes, and/or nanocomposites. The electrodes may be cellulose based, e.g. cellulose nanofibre. The electrodes may have a porous framework or comprise flexible materials.

Moreover, where the electrochemical cell comprises a fuel cell or biofuel cell, the electrochemical cell may comprise one or more catalysts selected at least in part based on said fuel. Catalysts may be selected based on the specific fuel or substrate, and may be selected based on or comprise attributes such as: being low cost, renewable, biodegradable and/or stable at room temperature. The catalyst(s) may be in solution or in a suspension, or fixed to electrode(s) to increase cell efficiency.

Where the electrochemical cell comprises a biofuel cell and/or an enzymatic biofuel cell, it may be configured to generate electrical energy in the presence of a fuel or substrate.

The electrochemical cell, in particular when comprising an enzymatic biofuel cell may comprise at least one enzyme. In some examples, either the at least one cathode and/or the at least one anode may comprise, carry, contain or be associated with at least one enzyme. Enzyme(s) may be immobilised on the electrode(s). The at least one enzyme may generally be configured to catalyse an oxidation of the fuel or substrate at the anode and/or catalyse a reduction of the oxidant at the cathode to thereby generate electrical energy.

Broadly, to endure mechanical deformations, the enzymatic biofuel cell may present physical characteristics of flexibility and/or stretchability, may be printable, and/or may be at least partly comprised of flexible materials e.g. fabric or cellulosic materials.

In some example enzymatic biofuel cells, only the anode may incorporate an enzyme for catalysis of the oxidation reaction at the anode, and the cathode may or may not include an enzyme that catalyses the reduction reaction of the oxidant.

In an example enzymatic biofuel cell, the at least one enzyme may be selected at least in part based on said fuel or substrate.

In an example enzymatic biofuel cell at least one oxidant may also be selected at least in part based on said fuel or substrate.

In another example enzymatic biofuel cell, the at least one anode comprises at least one first enzyme and the at least one cathode comprises at least one second enzyme. The first and second enzyme(s) may be the same or different enzyme. The at least one first enzyme may comprise a plurality of enzyme(s) the same or different to one another, and the at least one second enzyme may comprise a plurality of a plurality of enzyme(s) the same or different to one another.

The terms ‘first’ and ‘second’ when used herein in relation to enzyme(s) may be used solely to distinguish an enzyme associated with the cathode from another associated with the anode or vice versa, without necessarily requiring or implying any actual relationship or order between said enzyme(s).

Where such an example enzymatic biofuel cell is employed, the at least one first enzyme may be configured to catalyse an oxidation of the fuel at the anode and the at least one second enzyme may be configured to catalyse a reduction of the oxidant at the cathode to thereby generate electrical energy.

It will be appreciated that in some configurations, non-enzyme catalysts may be employed at the anode and/or cathode. Combinations of enzyme(s) and/or other catalyst(s) may be employed at the cathode and/or anode, to achieve desired redox reactions. Selection of enzyme(s) and/or other catalyst(s) may be based at least in part on a selected fuel.

In the example enzymatic biofuel cell at least one oxidant may also be selected at least in part based on said fuel or substrate.

The fuel or substrate may comprise a naturally occurring molecule or biological substrate. A wide range of sources can be used for the fuel. The fuel can be selected based at least in part on one or more of: availability, biodegradability, biocompatibility/possible toxicity, energy density and/or cost.

The fuel may for example comprise a hydrocarbon based fuel or substrate. The fuel may consist of any one or more of: sugar, glucose, pyruvate, lactose, lactic acid, alcohol, (e.g. ethanol, methanol, glycerol), starch and/or mixture(s) thereof. Glucose may be employed due at least in part to its high theoretical energy density (also since glucose is naturally occurring in the body, and so presents low toxicity).

The fuel(s) may be a liquid, a solid and/or a gel comprising at least one organic molecule. The at least one organic molecule may comprise any one or more of: sugar, glucose, pyruvate, lactose, lactic acid, alcohol, (e.g. ethanol, methanol, glycerol), starch and/or mixture(s) thereof.

Therefore, as the biofuel cell or enzymatic biofuel cell may be part of, or attachable to, the fixation structure operable in proximity to the patient, it is advantageous that the fuel comprise a non-toxic and/or biocompatible naturally occurring molecule or biological substrate, such as those listed above. These can be considered a safe, easy to handle, generally non-toxic, and biodegradable fuel that can safely be placed proximal, and/or contact/adhere to a patient's skin for extended periods of time.

Where the fuel or substrate is selected based on biocompatibility, it may be employed without triggering an immune response or causing toxicity in either the patient or medical personnel.

Where the fuel or substrate is selected based on energy density and/or cost, it may generate a high amount of electrical energy over a small footprint. Such fuel types may be a renewable resource that can be obtained from a variety of sources, are inexpensive and widely available, and thereby may reduce unit cost and environmental impact.

It will be appreciated that the term ‘enzyme’ when used herein may hence refer to any enzymatic system(s) which comprise a set of molecules and proteins allowing the catalysis of oxidation-reduction reactions which are carried out at the anode and at the cathodes.

It will be appreciated that such enzymatic biofuel cells may present no requirements for an external power source, provide relatively easy miniaturisation, may be renewable and/or biodegradable and present low toxicity.

In some examples, a power source may comprise a hybrid fuel cell, comprising a combination of biocatalysts at the anode and an inorganic catalyst at the cathode.

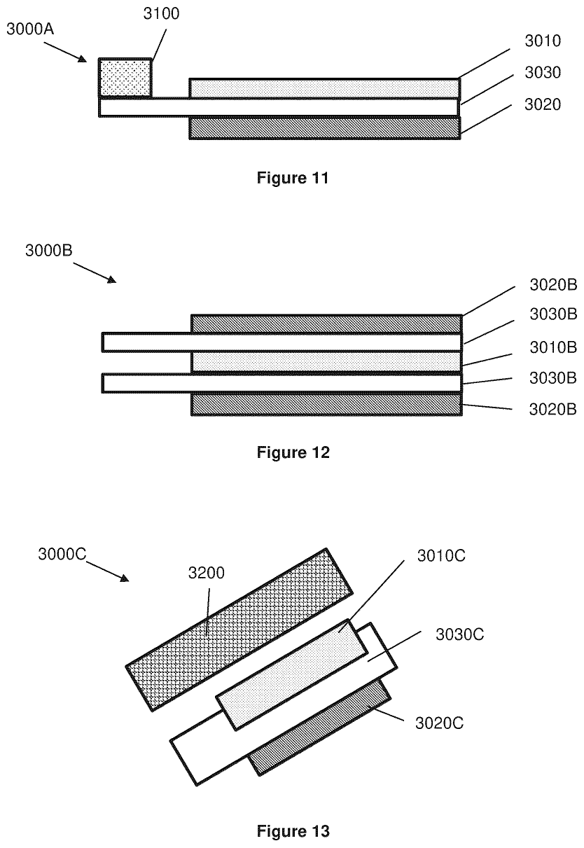

FIGS. 11 to 13 shows exemplary electrochemical cells 3000 that may be employed in the fixation structure to provide power to the sensor componentry. These electrochemical cells may be fuel cells or biofuel cells or enzymatic biofuel cells.

FIG. 11 shows a first example electrochemical cell 3000A having an anode 3010, a cathode 3020 and a reservoir 3100. The reservoir 3100 may comprise the fuel(s) or substrate(s). Where said first example electrochemical cell 3000A comprises a biofuel cell or enzymatic biofuel cell, said fuel(s) may comprise those described above. The fuel(s) inside the reservoir 3100 may be a liquid, a solid and/or a gel.

The reservoir 3100 may be configured to transfer the fuel contained therein to the at least one anode 3010 and/or at least one cathode 3020. In this respect, the at least one reservoir 3100 may be configured to be breakable, pierceable, and/or deformable so as to release the fuel contained therein to the at least one anode 3010 and/or at least one cathode 3020.

FIG. 11 also shows the example electrochemical cell 3000A having at least one membrane 3030 arranged between the at least one anode 3010 and the at least one cathode 3020.

The at least one membrane may generally be configured at least as a medium for facilitating electrochemical reactions between the anode and cathode.

The reservoir and its contents may, therefore, participate in the transition from an inactive to an active state of the electrochemical cell. The deformation of the reservoir and the release of its contents may in some examples facilitate or make possible an activation of the electrochemical cell, if not directly triggering said activation. For example, the release of the reservoir may create an intermediate activation state of the electrochemical cell.

Activation of the electrochemical cell may be direct or it may occur in several steps. The reservoir may be configured to be at least partially flexible and/or deformable.

In the example of FIG. 11, the at least one membrane, being a single membrane 3030, is provided as a medium for transfer of the fuel from the reservoir 3100 to the at least one anode and the at least one cathode.

Once the reservoir is activated to release the fuel therein, said fuel may pass through the at least one membrane 3030, i.e., via diffusion or absorption into the membrane, to reach the anode 3010 and cathode 3020, upon which, redox reactions may commence across the membrane and anode and cathode, in the presence of the now transferred fuel.

For this reason, the at least one membrane 3030 may be liquid permeable, porous and/or conductive.

The membrane 3030 may be liquid permeable, porous and conductive in the example biofuel cell 3000A of FIG. 11 in that the fuel contained in the reservoir 3100 may be provided as a liquid and/or gel. Hence, once released for transfer into and through the membrane 3030, from the location of the reservoir to/towards the anode and cathode, the liquid permeable, porous and conductive properties of the membrane 3030 facilitate the liquid and/or gel fuel to contact the anode and cathode and facilitate the redox reactions to occur across the membrane and resulting conduction of electrical power.

The at least one membrane may comprise a cellulose material, or may comprise of cellulose fibres. The at least one membrane may be or comprise at least one sheet or layer of paper, and in particular porous blotting paper and/or filter paper.

FIG. 12 shows a further example electrochemical cell 3000B, where like features to the example electrochemical cell 3000A are associated the same reference numerals but for the addition of ‘B’. For example, the electrochemical cell 3000B is shown with a central anode 3010B, flanked by two membranes 3030B on opposing sides thereof, with said membranes 3030B also flanked each by a respective cathode 3020B.

In this respect, electrochemical cell 3000B illustrates a multi-layered electrochemical cell having no reservoir but two membranes 3030B and two cathodes 3020B. The membranes 3030B may in this instance carry or be imbued with fuel, i.e., configured as mediums for storage of the fuel and/or mediums that comprise the fuel itself.

The membranes 3030B may be void of fuel but instead merely serve as mediums suitable for transfer of the fuel to the central anode 3010B and two cathodes 3020B. For example, a fuel may be selectively deposited onto the membranes 3030B, upon which they may transfer said fuel to the anode and cathodes to begin the redox reactions.

FIG. 13 shows a further example electrochemical cell 3000C, where again, like features to the previously described electrochemical cell 3000A, 3000B are associated the same reference numerals but for the addition of ‘C’.

The example electrochemical cell 3000C is shown in exploded perspective view as comprising one anode 3010C, one cathode 3020C, and one membrane 3030C. Here, no reservoir 3100 is provided. The membrane 3030C may serve as a medium for transfer of the fuel, a medium for storage of the fuel, and/or comprise or contain the fuel itself in this example.

The example electrochemical cell 3000C is shown having a protective layer 3200. The protective layer 3200 may be a solid cover, an at least partially flexible and/or deformable solid cover, and/or a coating. In this and other examples, the at least one protective layer may be at least partially flexible, non-toxic, chemically stable, fluid-impermeable and/or temperature and/or electrically insulating.

In this way, the protective layer 3200 may provide various protective benefits to the electrochemical cell 3000C, such as preventing contamination, water-damage, general wear, mis-use and/or mis-activation of the electrodes or membrane of the cell or the cell as a whole.

The protective layer 3200 may at least partially cover the electrochemical cell 3000C.

In some examples, the at least one protective layer may not completely surround the electrochemical cell so as to permit access of fuel and/or gas to the electrodes and membrane 3030C.

In this respect, when an at least one protective layer is employed, it may be configured to permit exposure of the electrochemical cell to the surrounding environment. For example, it may be configured such that fluid communication, may occur between the electrochemical cell and the ambient/external environment as desired.

In some examples, the at least one protective layer may comprise an adhesive layer, which may be water-resistant, allowing it to adhere to the external surface of the biofuel cell.

It should be noted that generally, maximising the surface area of the electrodes may improve the power output or battery life of a given electrochemical, such as biofuel cell. However, the consequent increase in footprint is undesirable in certain applications.

Hence, maximising surface area of the electrodes via multiple layers of electrodes superimposed upon one another, so as to maintain a minimal overall footprint of a given electrochemical cell may be advantageous in a patient interface setting, where it may be desirable to improve the performance of electrochemical cells, while avoiding or minimizing the increase in surface area, volume and/or mass of the device.

In this respect, multi-layer arrangements, such as shown in FIGS. 11 to 13 illustrate one advantageous means of providing maximised electrode surface area.

In some examples, the electrochemical cell is provided in or on a multi-layer arrangement. The electrochemical cell may comprise a plurality of layers and/or form at least part of the multi-layer arrangement.

An electrochemical cell may comprise a small profile and footprint that allow their integration into the fixation structure(s) described herein without adding substantial size or weight thereto.

It may be advantageous for an electrochemical cell to be at least partially flexible and/or deformable so as to suit the corresponding flexing and deforming of the fixation structure and features thereof when employed to affix a patient interface to the varying undulating surface of a patient's face.

In this respect, one or more of the at least one membrane, at least one protective layer, at least one reservoir and at least one electrode of a given electrochemical cell and aforementioned material(s) and/or make-up thereof may be at least partially flexible and/or deformable.

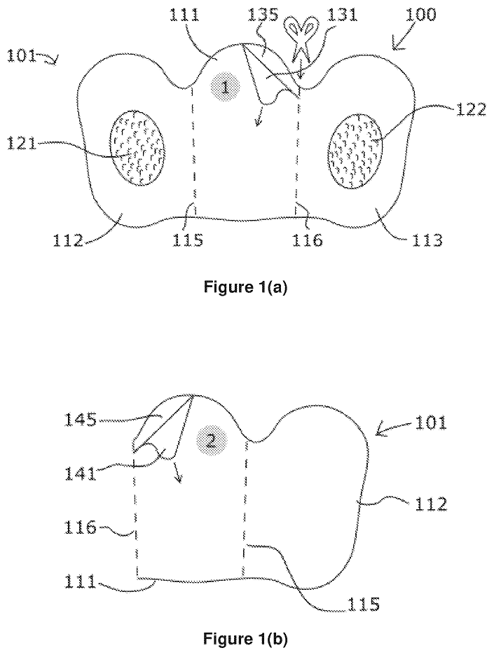

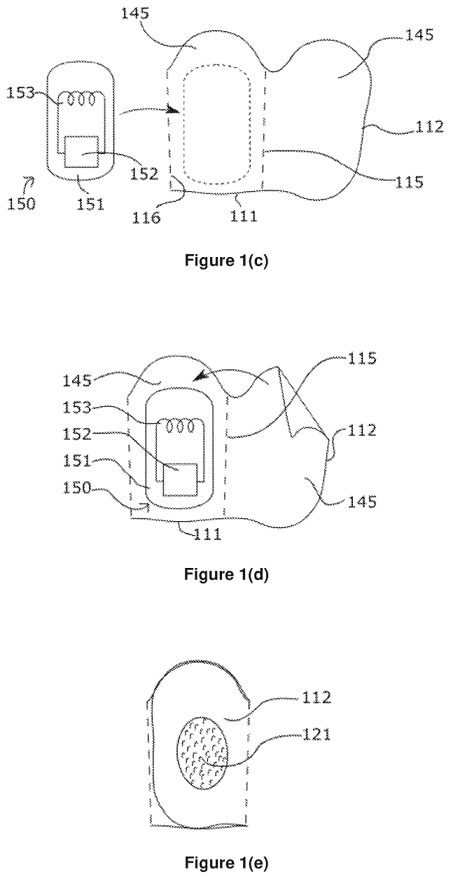

A given electrochemical cell may be integrated as part of the arrangement of the fixation structures described in relation to FIGS. 1A to 6 herein.

For example, a given electrochemical cell may have one or more of its layers, or its entirety, incorporated into the fixation structure.

The electrochemical cell may be provided on or in one or more of the body, the patient attachment and the fixing element or attachable to one or more of the body, the patient attachment and the fixing element.

Additionally or alternatively, the electrochemical cell may be attachable to or integrally formed with the first side of the body, the second side of the body, the patient attachment and/or the fixing element.

In this respect, the electrochemical cell may be provided to power the sensor componentry and hence be associated with the positioning or arrangement of the sensor componentry relative the fixation structure, i.e., relative the one or more of the body, the patient attachment and the fixing element or attachable to one or more of the body, the patient attachment and the fixing element.

An example electrochemical cell may be at least partly located between the body and the patient attachment, and/or at least partly encapsulated within the patient attachment. An electrochemical cell may be at least partly located between the body and the fixing element.

In some examples, a electrochemical cell may form a unitary structure with the fixing element and/or may be embedded within the fixing element.

The electrochemical cell may form part of the sensor componentry, and may be arranged relative the features of the fixation structure depending on the corresponding arrangement of the sensor componentry.

It will be appreciated that the electrochemical cell may be spaced apart and separate from the sensor componentry, and instead transmit power thereto via intermediate circuitry.

The electrochemical cell may comprise, or be connected to/in communication with, circuitry and a wireless emitter that wirelessly transmits power to a corresponding receiver coil of the sensor componentry.

Where an electrochemical cell is present, the wireless module(s) described herein may be powered via the same source of energy as the sensor componentry, i.e., by the electrochemical cell, or may be powered via the same source of energy as the sensor componentry, i.e., any one or more of the other sources of power described herein.

Other arrangements may be provided such that the sensor componentry is powered by energy harvested from an electrochemical source.

### Types of Sensors

The sensor componentry may comprise one or more sensors, for example, that may be included as sub-components of the sensor componentry. The one or more sensors can be configured to detect one or more patient parameters, such as, for example, physiological vital signs and/or blood chemistry parameters, environmental, usage and/or system parameters. Data from the sensor(s) may be used to evaluate the patient's condition. For example, conditions or parameters that may be detected include: apneas, perfusion levels, patient movement, pulse rate, respiratory rate, heart activity (e.g. ECG), brain activity (EEG), oxygen saturation (SpO₂), partial pressure of oxygen (pO₂), partial pressure of carbon dioxide (pCO₂).

One or more sensors of the sensor componentry can include, for example, one or more of skin electrode, photodetector, photoemitter, strain sensor, piezoresistor, thermistor, thermocouple, accelerometer, enzyme-based biosensor.

Measurements taken by the sensor(s) may be communicated to the therapy system or a device within the therapy system and may be used to alter therapy parameters. Alternatively or additionally, measurement data can be communicated to a patient monitoring system or device that records patient data. The patient monitoring system may signal an alarm when one or more parameters are outside of a predetermined range or which exceed or fall below a given threshold. The alarm may warn the patient, medical staff and/or a caregiver of any issues with the patient.

Parameter data taken by the sensor componentry(s) may be useful in a variety of situations, such as during therapy delivery (respiratory therapy, ventilator, infusion pump) or post-surgery monitoring. The fixation structure(s) as described herein can be used with any type of respiratory therapy, such as high flow, CPAP, BiLevel pressure therapy, Non-invasive positive pressure ventilation (NIPPV) therapy, low flow oxygen delivery or ET tubes, as non-limiting examples. The sensor componentry may comprise or connect to a memory to store parameter data from one or more sensor. The fixation structure may comprise the memory or the memory may be attached to the fixation structure or be part of the sensor componentry.

A temperature sensor can be included in the sensor componentry to measure, for example, the temperature of the patient. The temperature sensor may comprise any one or more of a thermistor, a resistance temperature detector (RTD), electrodermal activity (EDA) sensor, pyroelectric detector or a thermocouple. A pyroelectric detector may be suitable for use on a fixation structure if it is intended to be used over an extended period of time or if the sensor componentry is to be reusable. The passive temperature sensor may be a thermistor which requires a current applied to measure the voltage. A change of temperature may cause a change of resistance without requiring an external power source. The temperature sensor(s) used may be passive, such that they do not require a continuous power supply. This may make them small and relatively easy to integrate into the fixation structure. The temperature sensor may take periodic readings, instantaneous readings, or record a maximum and minimum temperature over a given/predetermined time period. The sensor componentry may transmit the temperature data as it is received from the temperature sensor or may store it in a memory for transmission at a later stage. The temperature data may be transmitted, for example, using Bluetooth, NFC, RFID or Wi-Fi to a mobile device or other receiving device.

A colour sensor may be used, for example, to record the colour of the patient's skin. The colour sensor readings may be taken at predetermined time intervals, such as every 10 minutes or every hour, as change in a patient's skin colour is unlikely to be rapid. If the colour sensor is used at predetermined intervals, the power usage of this sensor may be minimal.

Another type of sensor that may be provided is a thermochromic film, which may change colour based on temperature, such as the skin temperature of the patient. Alternatively or additionally, a film may be provided that changes colour based on CO₂ concentrations in the patient's blood (PCO₂). A colour sensor on the fixation structure may be used to sense the colour of the film to provide a measurement of the temperature or CO₂ concentration of the patient.

The sensor componentry may be provided with a motion sensor. The motion sensor may be utilised to detect when the patient is moving. This motion data may be used to determined when a patient is awake or asleep, or when a patient is attempting to leave a bed, for example.

A pulse oximetry sensor may be provided in the sensor componentry. One or more electronic components for reflectance pulse oximetry may be disposed on the fixation structure. For example they may be provided on a patient facing side of the fixation structure, such as on a patient facing side of a PCB. For example, a red (640 nm-700 nm) LED, an infrared (˜780 nm-1 mm) LED, and at least one light sensor (such as a photodiode). The pulse oximetry sensor may measure the saturation of oxygen in the patient's blood, ie may measure SpO₂ levels. An SpO₂ sensor may be used, for example, for closed loop oxygen titration.

As part of providing a patient with respiratory support, according to the present disclosure one or more parameters of the patient and/or of the respiratory support system may be measured by sensor componentry for the purpose of monitoring the patient's health. For example, a pulse oximeter may provide information relating to heart rate and blood oxygen saturation (SpO₂) of the patient. Sensors that monitor patient physiological conditions may also be used to provide input to the patient therapy device. For example, there is no specific prescribed level of supplemental O₂ that can consistently achieve an SpO₂ response in a targeted range for a given patient and it may be, therefore, useful to monitor SpO₂ and adjust supplemental O₂ accordingly. In the example of SpO₂, patients may benefit from having their fraction of oxygen delivered to the patient (FdO₂) monitored and adjusted to ensure they are receiving the correct FdO₂ to achieve targeted SpO₂.

FIG. 8 provides a schematic view of an example of a feedback loop. In this example, patient parameter information can be compared to target values to adjust therapy provided to the patient. This is one example of how the fixation structure and sensor componentry can be used. The present disclosure should be understood as being broader than the example shown in FIG. 8 and it is not restricted to use with a feedback loop control system. For example, one configuration may comprise a fixation structure for affixing a patient interface to a patient, the fixation structure having or being attachable to sensor componentry that senses at least one parameter, be that a parameter of the patient, the therapy system or an ambient parameter. The parameter data sensed by the sensor componentry may then be used for any desired purpose. For example, the data may be compared to a predetermined value(s) and an alert signalled when outside a desired range or above or below a threshold, or may be readable by a person, such as the patient or a caregiver, to provide them with an update of the sensed parameter(s), or any other desired purpose.

With reference to the example of FIG. 8, a pulse oximeter may provide information relating to heart rate and blood oxygen saturation (SpO₂). The closed loop control system shown in FIG. 8 may utilize two control loops. The first control loop may be implemented by the SpO₂ controller. The SpO₂ controller can determine a target FdO₂ based in part on the target SpO₂ and/or the measured SpO₂. The target SpO₂ value can be a single value or a range of acceptable values. The value(s) may be pre-set, chosen by a clinician, or determined automatically based on client characteristics. Generally, target SpO₂ values are received or determined before or at the beginning of a therapy session, though target SpO₂ values may be received at any time during a therapy session. During the therapy session, the SpO₂ controller can also receive as inputs: measured FdO₂ reading(s) from a gases composition sensor, and measured SpO₂ reading(s) and a signal quality reading(s) from the patient sensor. In some configurations, the SpO₂ controller can receive target FdO₂ as an input, in such a case, the output of the SpO₂ controller may be provided directly back to the SpO₂ controller as the input. Based at least in part on the inputs, the SpO₂ controller can output a target FdO₂ to the second control loop.

During the therapy session, the SpO₂ and FdO₂ controllers can continue to automatically control the operation of the respiratory support apparatus until the therapy session ends or an event triggers a change from the automatic mode to manual mode. For example, a respiratory support system using blood oxygen saturation measurements from a pulse oximeter to automatically adjust the fraction of oxygen of the gases flow being delivered to a patient via a patient interface is described in PCT publication no. WO2019/070136 of the present applicant and hereby incorporated by reference in its entirety.

A sensor may be used to measure the respiratory rate of the patient. The sensor may detect the occurrence of apnea in a patient. An SpO₂ sensor may be utilised for screening for apneas. With sufficient resolution, a pulse oximetry sensor may be used to measure the respiratory rate of a patient. The pulse oximetry sensor may be used to measure a heart rate of the patient.

In some configurations, the sensor componentry may comprise one or more electrodes to make an ECG measurement of the patient.

A PCO₂ sensor may measure a partial pressure of carbon dioxide of the patient. A PvCO₂ (partial venous pressure) sensor may be a suitable type of sensor for inclusion in a fixation structure.

In some configurations, two fixation structures or two sections of a separated fixation structure (for example, one structure on either side of the patient's face) may comprise sensor componentry comprising a sensing circuit. The structure on each side of the face may measure a different property of the patient. Alternatively or additionally, the two sides may measure the same, similar or linked properties to obtain a more accurate reading by averaging the values from each structure. This use of a fixation structure on either side of the patient's face may also be utilised to determine measurement errors. For example, a heart rate of the patient may be measured on either side of the patient's face.

In some configurations, a sensor provided on the interface side of the fixation structure may take readings relating to the therapy delivery. For example, the sensor may detect the temperature of delivered gas, a gas flow rate and/or pressure.

In some configurations, the circuitry of the sensor componentry may comprise a memory and be used to store information. The memory may store a patient identifier information. Identification or device information may be stored such that useful data is conveyed. Such data may include, for example, any of: type of patient interface device being used, patient interface size and/or the duration that the patient interface has been in use for. This information may be used to drive other parameters on the therapy device and/or may be used by care givers to assess when a change of the patient interface or other elements of the therapy system may be appropriate. Respiratory devices such as a patient interface may have a fixed usage period and the sensor componentry may be used to ensure that the device is not used beyond that period. For example, some interface devices may be used for a period of, for example, up to about 7 days before needing to be replaced by a new interface.

In some configurations, a scoring system may be used that is a combination of various sensed parameters which can provide an indication of the wellbeing or distress level of the patient. The therapy device may change therapy parameters based on the score detected.

Sensor readings, whether from one or a plurality of fixations structures each having at least one sensor componentry in different locations can be monitored to alert medical staff to any concerning conditions.

### EXAMPLES

FIG. 1(a) shows a first side of an example fixation structure 100 comprising a body 101 with a central main region 111, a first extension 112 and a second extension 113. In this example, the first extension 112 and second extension 113 are attached to the main region 111. The first extension 112 is connected to the main region 111 on a first edge 115. The second extension 113 is connected to the main region on a second edge 116. The main region 111, first extension 112 and second extension 113 may be formed unitarily as a single element. Alternatively, the main region 111, first extension 112 and second extension 113 may be formed as separate elements which may be joined together along the first edge 115 and second edge 116.

The body 101 may be formed from any suitable material. In other words, the main region 111, first extension 112 and/or second extension 113 may be formed from any suitable material. Each may be formed from the same material. The body material may be flexible. The body 101 may be formed, for example, from a polymer, such as polymer resin, polymer plastic and/or composite. The polymer may be thermoplastic. The body 101 may comprise, for example, polyurethane material or polyimide material. One example of a polyimide material is a polyimide film sold under the brand name Kapton.

The material forming the body 101 may be porous or at least partially porous. The material forming the body 101 may be breathable. The material forming the body 101 preferably has a moisture vapour transmission rate (MVTR) >0 g/m²/24 h. The MVTR may be at least about 100, 200, 400, 1000, 1500, 2000, 5000 or 10,000 g/m²/24 h.

In use, the first side of the main region 111 faces towards skin of the user/patient, i.e. the person to whom the fixation structure is to be attached. On the first side of the main region 111 there is provided a patient attachment 135 that permits the first side of the main region 111 to be adhered to the skin of a user/patient.

The patient attachment 135 may comprise an adhesive. Depending on the desired use and position of sensor componentry 150 in relation to the main region 111 of the fixation structure 100, the adhesive may be either conductive or non-conductive. The adhesive may be moisture permeable and/or porous. The adhesive may be dermatologically sensitive such that the skin of the user/patient may be less likely to have an adverse reaction to contact with the adhesive. The adhesive may include one or more of: a hydrocolloid-based adhesive material, a zinc oxide-based adhesive material, a silicone-based adhesive material, and/or a hydrogel-based adhesive material.

According to the example shown in FIG. 1(a)-(e), the first side of the main region 111 has a removable cover layer 131. The cover layer 131 is optional and its use may be dictated by the type of patient attachment 135 used. In the example shown in FIG. 1(e) the cover layer 131 is provided on the main region 111 over the patient attachment 135 such that the patient attachment 135 is protected from the external environment until the cover layer 131 is removed. The cover layer 131 may be formed from a suitable material that permits easy removal from the patient attachment 135. For example, where the patient attachment 135 is an adhesive, the material of the cover layer 131 is selected such that it will not permanently adhere to the adhesive and can contact and be removed from the patient attachment 135 without greatly affecting the adhesive properties of the patient attachment 135. The cover layer 131 may be a sheet or film of material comprising any of paper, plastic or polymer.

A fixing element 121 is located on the first side of the first extension 112. Similarly, a fixing element 122 is located on the first side of the second extension 113. The fixing elements 121, 122 each comprise a mechanical fastening element that attaches to a corresponding mechanical fastening element on a patient interface or cannula device. The fixing elements 121, 122 may each comprise one of a hook or loop material. For example, where the fixing elements 121, 122 comprise a hook material, the corresponding fastening element on the patient interface or cannula device will comprise a loop material. Alternatively, where the fixing elements 121, 122 comprise a loop material, the corresponding fastening element on the patient interface or cannula device will comprise a hook material. The fixing elements 121, 122 permit attachment to and removal from the corresponding fastening elements on the patient interface or cannula.

Either of the first extension 112 or second extension 113 may be detached from the main region 111 along the first edge 115 or second edge 116. For example, the first extension 112 or second extension 113 may be removed from the main region 111 by cutting along the respective first edge 115 or second edge 116, such as by using scissors or another sharp implement. Alternatively, the first edge 115 and/or second edge 116 may be weakened compared to the rest of the body 101 such that the first extension 112 or second extension 113 can be torn from the main region 111. The first edge 115 and/or second edge 116 may comprise a thinned material. The first edge 115 and/or second edge 116 may comprise perforations in the material of the body 101.

FIG. 1(b) shows the second side of the fixation structure 100 when the second extension 113 has been removed from the main region 111 along the second edge 116. In other words, FIG. 1(b) shows the main region 111 connected to only the first extension 112 along the first edge 115. An adhesive 145 is present on the second side of the body 101. In other words, adhesive 145 is provided on the second side of the main region 111. Adhesive 145 is provided on the second side of the first extension 112. Adhesive 145 is provided on the second side of the second extension 113. In some embodiments, the adhesive 145 may be provided on only one or more than one of the second side of the main region 111, the second side of the first extension 112 and the second side of the second extension 113. The adhesive 145 may be the same as or have the same properties as described above in relation to the attachment material 135. In some embodiments, the adhesive 145 may be provided on the second sides of the first extension 112 and second extension 113.

The second side of the body 101 comprises a cover material 141 that covers the first extension 112 and main region 111. The cover material 141 may also cover the second side of the second extension 113. The cover material 141 may comprise weakened regions in the locations corresponding to the first edge 115 and second edge 116 to assist in the removal of the first extension 112 or second extension 113. For example, the cover material 141 may be perforated along the first edge 115 and second edge 116. The cover material 141 may be the same as or have the same properties as the cover material 131 as described above.

The adhesive 145 is provided such that, when the cover material 141 has been removed, one of the first extension 112 or second extension 113 may be folded over onto the main region 111. When folded over the second surfaces of the first extension 112 or second extension 113 adheres to the second surface of the main region 111. The first extension 112 may pivot or fold relative to the main region 111 along the first edge 115. The second extension 113 may pivot or fold relative to the main region 111 along the second edge 116.

As shown in FIG. 1(c), sensor componentry 150 is provided with the fixation structure 100. In this example, the sensor componentry 150 is a separate element to the structure of the body 101 and is configured to be attached to the body 101 when one of the first extension 112 or second extension 113 is folded over onto the main region 111. FIG. 1(d) depicts the second side of the main region 111 and first extension 112 when the cover material 141 has been removed and the sensor componentry 150 is positioned in the centre of the second side of the main region. The sensor componentry 150 is held in place by the adhesive 145. As also shown in FIG. 1(d), the second side of the first extension 112 is then folded over onto the second side of the main region 111 and onto the sensor componentry 150. The sensor componentry is then sandwiched between the second sides of the main region 111 and first extension 112. A similar sandwiching arrangement could be provided with the sensor componentry 150 between the second sides of the main region 111 and second extension 113 if the second extension 113 has not been removed. FIG. 1(e) depicts the resulting fixation structure when the steps (a) to (d) have been followed and the sensor componentry 150 is held between the main region 111 and first extension 112. In this arrangement, the first side of the main region 111 can be attached to the skin of the user/patient and the first side of the first extension 112 points away from the user/patient with the fixing element 121 available for attachment to the corresponding patient interface or cannula device.

The fixation structure 100 shown in FIG. 1(a)-(e) may further be used to hold a tube, such as a nasogastric (NG) tube in position relative to the skin of the user/patient. For example, the NG tube may be placed longitudinally along the fold/pivot line (first edge 115 or second edge 116) on the second side of the body 101 after the cover layer 141 has been removed. The NG tube can be held in place by the adhesive 145 and/or between the second sides of the main region 111 and first extension 112 when they are folded over onto one another.

The sensor componentry 150 comprises a base substrate 151 to which the other elements of the sensor componentry 150 are attached. The base substrate 151 may be formed from a flexible material. The base substrate 151 may comprise a polymer material, for example, a urethane material. The base substrate 151 may be formed from the same material as used for the body 101 or having one or more of the attributes of the material of the body 101 as discussed above.

The sensor componentry 150 may include a power source. The sensor componentry 150 may comprise an antenna. In the example shown in FIG. 1(a)-(e), the sensor componentry 150 comprises a coil 153. The coil 153 may function as one or both of an antenna and/or a power source. The coil 153 is provided on the base substrate 151. The coil 153 may be printed directly onto the base substrate 151. The coil 153 may be formed at least partially by a conductive ink, which may be screen printed onto the base substrate 151. The coil 153 or parts thereof may be provided on the base substrate by photolithography, another lithographic technique or any other suitable process.

The sensor componentry 150 further comprises a sensor 152. The sensor componentry 150 may comprise more than one sensor 152. The sensor 152 may be provided as part of an integrated circuit or other electronic package. The sensor 152 is electronically connected to the coil 153. The coil 153 may send data that is sensed by the sensor 152.

The sensor componentry 150 may comprise a microcontroller. The microcontroller may have a memory that stores data sensed by the sensor 152. The memory may hold patient specific information, such as patient identifier information. The sensor 152 is provided on the base substrate 151. Electrical connections required for sensor 152 may be printed directly onto the base substrate 151. The electrical connections may be formed at least partially by a conductive ink, the conductive ink may be screen printed onto the base substrate 151. The electrical connections, in the form of tracks or traces of a PCB, or parts thereof may be provided on the base substrate by photolithography, another lithographic technique or any other suitable process.

The sensor 152 and coil 153 may be integral parts of the sensor componentry 150. According to alternative embodiments, the sensor componentry 150 may be provided on or in the body 101 directly. For example, the sensor componentry 150 may be provided directly on the body 101. The base substrate 151 may be attached to or provided on or in one or more of the main region 111, first extension 112 and/or second extension 113. The sensor componentry 150 may not comprise a separate base substrate 151 and may instead be attached to or printed directly onto the body 101.

The sensor componentry 150 may be powered via a suitable source of power as discussed above. For example, the coil 153 may inductively provide power from a magnetic field generated near to the fixation structure. Alternatively or additionally, the sensor componentry 150 may comprise a battery, such as a thin film battery. Alternatively or additionally, the sensor componentry 150 may be powered by an electrochemical cell, and/or may comprise said electrochemical cell, as described above. Additionally or alternatively, a sensor of the sensor componentry 150 may be a passive sensor not requiring a power provided by the sensor componentry itself but from an outside source when a reading is to be taken. Any other suitable means of powering the sensor componentry 150 or elements thereof may be utilised.

FIG. 1 (a)-(e) relates to one embodiment of which many adaptations to the structure are possible within the scope of the present disclosure. For example, a fixation structure may comprise a single extension or no extension. According to alternative embodiments, the extension that folds over onto the main region may be replaced by a separate layer that sandwiches together with the main region in a similar manner but without being hinged together. One or both of the main region and separate layer may comprise an adhesive to connect the main region and separate layer together in the sandwich arrangement. The sensor componentry may be placed in the between the main region and the separate layer before they are sandwiched together. In this embodiment, one of the main region and separate layer may comprise the patient facing side that can be removably attached to the skin of the patient and the other of the main region and separate layer may comprise the fixing element for removable attachment to the patient interface.

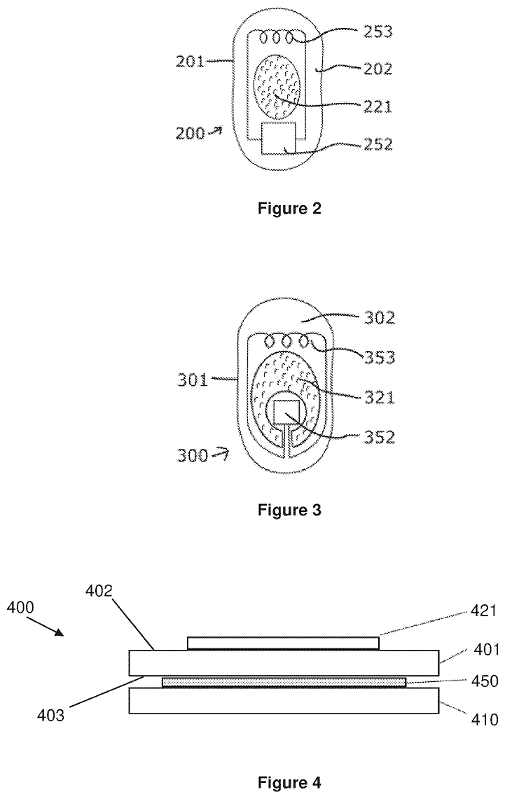

FIG. 2 shows a further example embodiment of a fixation structure 200 according to the present disclosure. The fixation structure 200 comprises a body 201 having a patient facing side (first side, not shown) and an interface facing side 202 (second side). The patient facing side is removably attachable to the skin of a patient. For example, the patient facing side may comprise an adhesive that permits the fixation structure to be easily attached to the patient's skin. The adhesive material used may be in accordance with any adhesive as disclosed herein.

The interface facing side 202 comprises a fixing element 221 or fixing element. The fixing element 221 in this embodiment is one of a hook or loop material and is configured to removably attach to a corresponding loop or hook material on the patient interface. Other means for fixing or fastening the fixation structure to the patient interface, as disclosed herein or otherwise, may be utilised in this embodiment. The fixing element 221 in this embodiment is shown as positioned in a central region on the interface facing side 202. The fixing element 221 may be positioned in a non-central region of the interface facing side 202 in other possible embodiments.

Surrounding the fixing element 221 on the interface facing side 202 are elements of the sensor componentry shown. The sensor componentry may be at least partly embedded in one or more layers of the fixation structure 200. The sensor componentry of this embodiment comprises a sensor 252. The sensor may be part of an integrated circuit or other electronics package. The sensor 252 is attached to a coil 253. The coil 253 may be used as an antenna and/or receiver to transmit or receive data, such as transmitting parameter data sensed by the sensor(s) of the sensor componentry. The coil 253 may alternatively or additionally be utilised to provide an inductive power to the sensor componentry. For example, in use the coil 253 may be placed perpendicularly to an externally applied magnetic field. The coil 253 may possess any feature or be utilised for any purpose as described herein in relation to a coil.

The fixation structure 200 shown in FIG. 2 has sensor componentry that may utilise a substantial portion, the majority or the whole of the available space on the interface facing side 202 that is not taken up by the fixing element 221. It may be beneficial that the coil 253 is as large as permitted by the embodiment. The size and arrangement of the sensor componentry may vary depending on the size of the fixation structure as a whole and the relative size of fixing element relative to surface area of the body on which it is positioned.

The sensor componentry of FIG. 2 may be provided on a base substrate, such as described in relation to FIG. 1(a)-(e). The base substrate of the sensor componentry in that case may be affixed to the interface facing side 202 of the fixation structure 200 by a suitable means, such as by an appropriate adhesive as discussed herein. Alternatively, one or more elements of the sensor componentry may be provided directly on the surface of the interface facing side 202 of the fixation structure 200. For example, the sensor 252 may be connected to electrical connections that are printed using a conductive ink on the fixation structure 200. The coil 253 may be printed directly onto the fixation structure 200, such as by using a conductive ink.

Any of the features of the embodiment of a fixation structure 200 according to FIG. 2 may be combined with any other aspect or embodiment of the present disclosure as described herein. For example, the sensor componentry according to the embodiment of FIG. 2 may comprise any of the features of wired or wireless communication, such as including a wireless module, or method of being powered as described herein. Alternatively, the sensor componentry could be provided in a location other than on the interface facing side of the fixation structure 200, as described herein.

FIG. 3 shows another example embodiment of a fixation structure 300 according to the present disclosure. The fixation structure 300 comprises a body 301 having a patient facing side (first side, not shown) and an interface facing side 302 (second side). The patient facing side is removably attachable to the skin of a patient. For example, the patient facing side comprises an adhesive that permits the fixation structure to be easily attached to the patient's skin. The adhesive material used may be in accordance with any adhesive as disclosed herein.

The interface facing side 302 comprises a fixing element 321. The fixing element 321 may be one of a hook or loop material and is configured to removably attach to a corresponding loop or hook material on the patient interface. Other means for fixing or fastening the fixation structure 300 to the patient interface, as disclosed herein or otherwise, may be utilised in this embodiment.

The sensor componentry of this embodiment comprises a sensor 352. The sensor may be part of an integrated circuit or other electronics package. The sensor 352 is connected to a coil 353. The coil 353 may be used as an antenna and/or receiver to transmit or receive data, such as transmitting parameter data sensed by the sensor(s) of the sensor componentry. The coil 353 may alternatively or additionally be utilised to provide an inductive power to the sensor componentry. For example, in use the coil 353 may be placed perpendicularly to an externally applied magnetic field. The coil 353 may possess any feature or be utilised for any purpose as described herein in relation to a coil.

The fixing element 321 may be positioned in a central region on the interface facing side 302. It is also possible that the fixing element 321 is positioned in a non-central region of the interface facing side 302. In this embodiment, the fixing element 321 could fill a substantial portion, the majority or the whole of the interface facing side 302 of the fixation structure 300. The elements of the sensor componentry may be positioned within or at least partially within the bounds of the fixing element 321. For example, the sensor 352 is positioned within the fixing element 321. The sensor componentry may be at least partly embedded within the fixing element 321. Embedding the sensor componentry within the fixing element 321 may provide at least some additional physical protection for the sensor componentry.

The coil 353 is positioned both inside and outside of the fixing element 321. In other words, the coil 353, extends outwardly from within and around the fixing element 321. This arrangement permits the coil 353 to utilise a large portion of the surface area of the interface facing side 302 of the fixation structure 300. The coil 353 using the maximum amount of surface area may be beneficial in the functioning as an antenna or as an inductive coil. The coil 353 could be provided entirely within the bounds of the fixing element 321. Alternatively, the coil 353 could be provided outside of the bounds of the fixing element 321.

In the embodiment shown in FIG. 3, the fixing element 321 comprises a hook material. The hook material protrudes out from the interface facing side 302 of the fixation structure 300. This means that the componentry of the sensor componentry embedded within the fixing element 321 may be provided with some protection by the protruding hooks.

The fixation structure 300 shown in FIG. 3 has sensor componentry that utilises a substantial portion of available space on the interface facing side 302 including space that is taken up by the fixing element 321. It may be beneficial that the coil 353 is as large as possible on the interface facing side 302 in this embodiment.

The sensor componentry of FIG. 3 may be provided on a base substrate, such as described in relation to FIG. 1(a)-(e). The base substrate of the sensor componentry in that case may be affixed to the interface facing side 302 of the fixation structure 300 by a suitable means, such as by an appropriate adhesive as discussed herein. One or more elements of the sensor componentry may be provided directly on the surface of the interface facing side 302 of the fixation structure 300. For example, the sensor may be connected to electrical connections that are printed using a conductive ink on the fixation structure 300. The coil 353 may be printed directly onto the fixation structure 300, such as by using a conductive ink.

Any of the features of the embodiment of a fixation structure 300 according to FIG. 3 may be combined with any other aspect or embodiment of the present disclosure as described herein. For example, the sensor componentry according to the embodiment of FIG. 3 may comprise any of the features of sensor componentry, wired and/or wireless module or method of being powered as described herein. Alternatively, the sensor componentry could be provided in a location other than on the interface facing side of the fixation structure 300, as described herein.

FIG. 4 shows a cross-sectional view of an example fixation structure 400 according to the present disclosure. The fixation structure 400 comprises a body 401 having a patient facing side 403 (first side) and an interface facing side 402 (second side). The patient facing side 403 is removably attachable to the skin of a patient. The patient facing side 403 comprises an adhesive layer 410 that permits the fixation structure 400 to be easily attached to the patient's skin. The adhesive material of the adhesive layer 410 may be in accordance with any adhesive as disclosed herein. For example, the adhesive may be a hydrocolloid or any other dermatologically appropriate adhesive suitable for securement to the skin of a patient.

The interface facing side 402 comprises a fixing element 421 or fixing element. The fixing element 421 may be one of a hook or loop material and is configured to removably attach to a corresponding loop or hook material on the patient interface. Other means for fixing or fastening the fixation structure 400 to the patient interface, as disclosed herein or otherwise, may be utilised in this embodiment.

The body 401 may be a film layer or substrate. The body 401 may be constructed of a suitable material as described herein. For example, the body 401 may be produced from a moisture permeable and/or porous material. The body 401 may be formed from a breathable material. The material of the body 401 may comprise a material with >0 g/m²/24 h MVTR which may allow breathability and/or may prevent or reduce build-up of moisture beneath the fixation structure 400. The breathability of the fixation structure may reduce moisture build-up between the body and the skin of the patient. The body 401 may be manufactured from a polymer material. In some configurations, the body 401 may comprise a material comprising one or more of urethane based polymer or imide based polymer. According to some configurations, the body 401 comprises a polyimide, for example polyimide film sold under the brand name Kapton.

Sensor componentry 450 in this example, is provided between the body 401 and adhesive layer 410. The sensor componentry 450 may comprise a flexible PCB. The sensor componentry 450 or PCB may be formed at least partly from one or more of imide-based polymer, ester-based polymer (polyester PET), PEN (polyethylene naphthalate), PTFE (Polytetrafluoroethylene) and/or Aramid (aromatic polyamide). The sensor componentry 450 may comprise a non-flexible PCB. The PCB may be formed at least partly from one or more of: dielectric composites, phenolic resin, epoxy, polyester, glass (fibres) and/or cotton paper. The PCB may be porous. The sensor componentry 450 or PCB may be manufactured from a polyimide. The sensor componentry 450 or PCB may comprise Kapton.

The sensor componentry 450 may be formed on a base substrate, such as by screen printing or any other suitable method. The base substrate may be formed from a polymer material, for example, a urethane material. The sensor componentry 450 may cover substantially all of the surface area of the patient facing side 403 of the body 401. Alternatively, the sensor componentry 450 may cover a limited portion less than the entirety of the surface area of the patient facing side 403 of the body 401. The sensor componentry 450 may be at least partly porous and/or breathable, which may assist with breathability of the fixation structure 400.

The sensor componentry 450 may be placed adjacent to the patient facing side 403 of the body 401. The adhesive layer 410 may be laid over the sensor componentry 450 and patient facing side 403 of the body 401. In that case, the sensor componentry 450 may be encapsulated within the adhesive layer 410 on the patient facing side 403. The sensor componentry 450 may be printed directly onto the patient facing side 403, such as by screen printing a conductive ink using any suitable process. The sensor componentry 450 could be provided directly on or in the adhesive layer 410.

The fixation structure of FIG. 4 may be combined with the features of a fixation structure as described in relation to any embodiment or aspect described herein. For example, the sensor componentry 450 of this embodiment may comprise a coil, a multi-layer PCB arrangement, a battery, and/or a wireless module, as examples of possible features. Any combination of the fixation structure 400 of this configuration with any other feature of a fixation structure described herein is contemplated by the present disclosure.

FIG. 5 shows a cross-sectional view of a fixation structure 500 according to a further example of the present disclosure. The fixation structure 500 of FIG. 5 has similar features to the fixation structure 400 of FIG. 4, other than the positioning of the sensor componentry 550 is different. The fixation structure 500 comprises a body 501 having a patient facing side 503 (first side) and an interface facing side 502 (second side). The patient facing side 503 is removably attachable to the skin of a patient. The patient facing side 503 comprises an adhesive layer 510 on the patient facing side 503 that permits the fixation structure 500 to be easily attached to the patient's skin. The adhesive material of the adhesive layer 510 may be in accordance with any adhesive as disclosed herein. For example, the adhesive may be a hydrocolloid or any other dermatologically appropriate adhesive suitable for securement to the skin of a patient.

The interface facing side 502 comprises a fixing element 521 or fixing element. The fixing element 521 may be one of a hook or loop material and is configured to removably attach to a corresponding loop or hook material on the patient interface. Other means for fixing or fastening the fixation structure 500 to the patient interface, as disclosed herein or otherwise, may be utilised in this configuration.

The body 501 may be a film layer or substrate. The body 501 may be constructed of a suitable material as described herein. For example, the body 501 may be produced from a moisture permeable and/or porous material. The body 501 may be formed at least partially from material that is breathable. The material of the body 501 may comprise a material with >0g/m²/24 h MVTR material which may allow breathability and/or may prevent or reduce build-up of moisture beneath the fixation structure 500. The body 501 may be manufactured from a suitable material, for example, a polymer, polymer resin, polymer plastic and/or composite. The body 501 may comprise, for example, polyurethane or polyimide material. One example of a polyimide material is a polyimide film sold under the brand name Kapton.

In the example shown in FIG. 5, sensor componentry 550 is provided on the interface facing side 502 of the fixation structure 500. The sensor componentry 550 shown in FIG. 5 is between the body 501 and the fixing element 521. The sensor componentry 550 may comprise a flexible PCB. The PCB may be porous and/or breathable. The sensor componentry 550 may be formed on a base substrate, such as by screen printing or any other suitable method. The base substrate may be formed from a polymer material, for example, a urethane material. The sensor componentry 550 may cover substantially all of the surface area of the interface facing side 502 of the body 501. The sensor componentry 550 may cover a limited portion less than the entirety of the surface area of the interface facing side 502 of the body 501. The sensor componentry 550 may be porous and/or breathable, which may assist with breathability of the fixation structure 500.

The sensor componentry 550 may be provided on a base substrate, such as a suitable polymer material, for example, a urethane material. The sensor componentry 550 may be placed adjacent to the interface facing side 502 of the body 501 and then fastener may be laid over and attached to the sensor componentry 550 and interface facing side 502 of the body 501. The sensor componentry 550 may be at least partly encapsulated within an adhesive on the interface facing side 502 between the body 501 and fixing element 521. The sensor componentry 550 may additionally or be provided directly onto the interface facing side 502. The sensor componentry 550 could be provided directly on or in the fixing element 521. The features of the example of FIG. 5 may be combined with the features of either of FIG. 2 or 3 with regards to placement of the fixing element 521 and features of the sensor componentry 550.

The example of FIG. 5 may be combined with the features of a fixation structure as described in relation to any configuration, example or aspect herein. For example, the sensor componentry 550 of this configuration may comprise a coil, a multi-layer PCB arrangement, a battery, and/or a wireless module, as examples of possible features. Any combination of the fixation structure 500 of this configuration with any other feature of a fixation structure described herein is contemplated by the present disclosure.

The position of the sensor componentry with respect to the fixation structure may be determined by the type of sensor used and the parameter to be detected. For example, some types of sensor may benefit from being located closer to the skin of the patient, such as on or in the patient facing side of the fixation structure or adhesive layer. Some sensors may benefit from being distanced from the skin of the patient or located in closer proximity to the patient interface, such as where the sensor detects parameters of the interface, such as flow rates etc.

Some modifications to the fixation structure may assist to facilitate improved or more accurate measurements by the sensor(s). With reference to FIG. 6, there is shown a further example of fixation structure 600. Similar to the example shown in FIG. 4, the fixation structure 600 includes a body 601 having a patient facing side 603 and an interface facing side 602. A fixing element 621 is located on the interface facing side 602. Sensor componentry 650 is positioned on the patient facing side 603 between the body 601 and an adhesive layer 610.

In contrast to the example of FIG. 4, the example of FIG. 6 has an aperture 611 provided in the adhesive layer 610. An aperture may be additionally or alternatively provided in one or both of the body 601 and the fixing element 621. In addition to or instead of an aperture, the embodiment of FIG. 6. The aperture 611 in the adhesive layer 610 corresponds in location to the position of a sensor 652 on the sensor componentry 650. The aperture 611 in the adhesive layer 610 provides an absence of adhesive material between the sensor 652 and the skin of the patient. This arrangement may be beneficial when the sensor 652 requires a clear line of sight to the skin of the patient to obtain an accurate reading. For example, the sensor 652 may include: a colour sensor to sense the colour of the patients skin; or a pulse oximeter.

For a pulse oximeter, the sensor comprises a red or IR light, such as an LED. The aperture 611 allows the light of the pulse oximeter to easily be directed to the patient's skin. The aperture 611 may alternatively be replaced by a thinned region of the one or more layers of the fixation structure 600. For example the fixation structure 600 may comprise at least one thinned region in any one or more of the adhesive layer 610, the body 601 and the fixing element 621.

According to other embodiments, the sensor componentry may be provided on the interface facing side of the body. A sensor of the sensor componentry may still require clearer access to the skin of a patient. In that case, an aperture or thinning of the adhesive layer and/or other layers of the body could be provided in combination with an aperture or thinning of the body. The fixation structure may comprise an aperture through one or more layers of its structure (adhesive, body/substrate, fixing element) in order to provide direct access to the skin of the patient to sensor componentry.

The example of FIG. 6 may be combined with the features of a fixation structure as described in relation to any example, configuration or aspect herein. For example, the sensor componentry 650 of this example may comprise a coil, a multi-layer PCB arrangement, a battery, and/or a wireless module, as examples of possible features. Any combination of the fixation structure 600 of this example with any other feature of a fixation structure described herein is contemplated by the present disclosure.

While the present disclosure has been described in conjunction with a limited number of configurations, it will be appreciated by those skilled in the art that many alternative, modifications and variations in light of the foregoing description are possible. Accordingly, the present disclosure is intended to embrace all such alternative, modifications and variations as may fall within the spirit and scope of the disclosure as disclosed.

Any reference to or discussion of any document, act or item of knowledge in this specification is included solely for the purpose of providing a context for the present invention. It is not suggested or represented that any of these matters or any combination thereof formed at the priority date part of the common general knowledge, or was known to be relevant to an attempt to solve any problem with which this specification is concerned.

In this specification, the terms ‘comprises’, ‘comprising’, ‘includes’, ‘including’, or similar terms are intended to mean a non-exclusive inclusion, such that a method, system or apparatus that comprises a list of elements does not include those elements solely, but may well include other elements not listed.

1. A fixation structure for securing a patient interface to a patient, the fixation structure comprising:
a body having a first side and a second side;
a patient attachment on the first side of the body;
a fixing element on the second side of the body; and
sensor componentry provided on or in one or more of the body, the patient attachment and the fixing element or attachable to one or more of the body, the patient attachment and the fixing element.

2. The fixation structure of claim 1, wherein the sensor componentry is at least partially flexible.

3. The fixation structure of claim 1 or 2, wherein the sensor componentry comprises a PCB.

4. The fixation structure of any one of the preceding claims, wherein the first side of the body comprises a patient attachment.

5. The fixation structure of any one of the preceding claims, wherein the patient attachment comprises an adhesive.

6. The fixation structure of claim 5, wherein the adhesive is dermatologically sensitive and comprises any one or more of: a hydrocolloid-based adhesive material, a zinc oxide-based adhesive material, a silicone-based adhesive material, and/or a hydrogel-based adhesive material.

7. The fixation structure of any one of the preceding claims, wherein the sensor componentry is at least partly located between the body and the patient attachment, is at least partly encapsulated within the patient attachment, or is at least partly located between the body and the fixing element.

8. The fixation structure of any one of the preceding claims, wherein the fixing element is positioned in a first region of the second side of the body and the sensor componentry is least partially positioned in a second region of the second side of the body.

9. The fixation structure of claim 8, wherein the second region surrounds the first region of the second side of the body and/or wherein the first region and second region at least partly overlap.

10. The fixation structure of any one of the preceding claims, wherein the sensor componentry forms a unitary structure with the fixing element and/or the sensor componentry is embedded within the fixing element.

11. The fixation structure of any one of the preceding claims, wherein the sensor componentry is provided in or on a multi-layer arrangement.

12. The fixation structure of claim 11, wherein the body comprises a plurality of layers and forms at least part of the multi-layer arrangement.

13. The fixation structure of claim 12, wherein the sensor componentry is provided on a plurality of the layers of the body.

14. The fixation structure of any one of the preceding claims, further comprising a wireless module.

15. The fixation structure of claim 14, wherein the wireless module comprises an antenna that transmits data sensed by the sensor componentry and/or a receiver to receive data, optionally wherein the wireless module comprises a transceiver to transmit and receive data.

16. The fixation structure of claim 14 or 15, wherein the wireless module transmits and/or receives data according to a wireless communications protocol, such as Bluetooth, Wi-Fi, BLE, RFID, ANT+ or near-field communication (NFC).

17. The fixation structure of any one of claims 14 to 16, wherein the wireless module is configured to receive a prompt from an external device to transmit data and to transmit data in response to said prompt.

18. The fixation structure of any one of the preceding claims, wherein the sensor componentry is powered wirelessly.

19. The fixation structure of claim 18, wherein the sensor componentry comprises a receiving coil that receives a power from an emitting coil.

20. The fixation structure of any one of claims 14 to 19, wherein the wireless module is powered via the same source of energy as the sensor componentry.

21. The fixation structure of any one of the preceding claims, wherein the sensor componentry receives power from an electrochemical cell.

22. The fixation structure of claim 21, wherein said electrochemical cell comprises a fuel cell and/or a biofuel cell.

23. The fixation structure of claim 21 or 22, wherein said electrochemical cell comprises an enzymatic biofuel cell.

24. The fixation structure of any one of claims 21 to 23, wherein the electrochemical cell comprises a fuel and at least one enzyme selected at least in part based on the fuel.

25. The fixation structure of claim 24, wherein the fuel comprises a hydrocarbon-based fuel or substrate.

26. The fixation structure of any one of the preceding claims, wherein the body is formed from an at least partially porous material and/or at least part of the fixation structure is breathable.

27. The fixation structure of any one of the preceding claims, wherein the body is formed from a material with a moisture vapour transmission rate of greater than 0 g/m²/24 h.

28. The fixation structure of any one of the preceding claims, wherein the fixing element is a mechanical fastener configured to attach to the patient interface.

29. The fixation structure of any one of the preceding claims, wherein the sensor componentry senses at least one parameter of the patient.

30. The fixation structure of claim 29, wherein the parameter is one or more of: temperature, skin colour, patient movement, CO₂ concentration in the patient's blood (PCO₂), saturation of peripheral oxygen (SPO₂), heart rate, heart rhythm, electrical activity and/or respiratory rate.

31. The fixation structure of any one of the preceding claims, wherein the sensor componentry comprises a temperature sensor, a motion sensor, an electrocardiogram (ECG) sensor and/or at least one LED.

32. The fixation structure of any one of the preceding claims, wherein the sensor componentry comprises a colour sensor configured to detect a colour of the skin of the patient and/or a pulse oximeter sensor that determines a saturation of peripheral oxygen (SPO₂) in the blood of the patient.

33. The fixation structure of any one of the preceding claims, wherein the sensor componentry comprises at least one film that changes colour in response to a change in a parameter of the patient, and wherein the film is a thermochromic film that changes colour in response to a change of temperature of the patient or changes colour to reflect a detected CO₂ concentration in the patient's blood (PCO₂).

34. The fixation structure of any one of the preceding claims, comprising an aperture, wherein a sensor of the sensor componentry is located in or adjacent to the aperture such that the sensor may sense a parameter of or through the skin of the patient when the fixation structure is attached to the patient and/or wherein the patient attachment comprises a thinned region where a thickness of the patient attachment is reduced, a sensor of the sensor componentry being located directly adjacent to the thinned region such that the sensor may sense a parameter of or through the skin of the patient when the fixation structure is attached to the patient.

35. The fixation structure of any one of preceding claims, wherein the body comprises at least a first layer and a second layer, and wherein the sensor componentry is at least partly sandwiched between the first layer and the second layer.

36. A fixation structure for securing a patient interface to a patient, the fixation structure comprising:
a body comprising a main region and a first extension, each of the main region and the first extension having a first side and a second side, wherein the first side of the main region is removably attachable to the patient;
a first fixing element on the first extension, the first fixing element is removably attachable to the patient interface; and
a first sensor componentry provided on or in one or both of the body, and the first fixing element or attachable to one or both of the body and the first fixing element,
wherein the first extension is attachable to the second side of the main region.

37. The fixation structure of claim 36, wherein:
the first fixing element is on the first side of the first extension;
the second side of the main region and second side of the first extension are attachable to one another; and
the first extension is pivotable relative to the main region from a first orientation to a second orientation where the second side of the first extension attaches to the second side of the main region.

38. The fixation structure of claim 37, wherein one or both of the second side of the main region and the second side of the first extension comprises an attachment configured to attach the second side of the main region and the second side of the first extension.

39. The fixation structure of any one of claims 36 to 38, wherein the body comprises a second extension having a first side and a second side, the first extension being attached to a first edge of the main region and the second extension being attached to a second edge of the main region and wherein the first edge and second edge of the main region are substantially parallel to one another.

40. The fixation structure of claim 39, wherein:
a second fixing element is on the first side of the second extension, the second fixing element being removably attachable to the patient interface.

41. The fixation structure of any one of claims 39 or 40, wherein:
the second side of the main region and the second side of the second extension are attachable to one another; and
the second extension is pivotable relative to the main region from a first orientation to a second orientation where the second side of the second extension attaches to the second side of the main region.

42. The fixation structure of claim 41, wherein one or both of the second side of the main region and the second side of the second extension comprises an attachment.

43. The fixation structure of claim any one of claims 39 to 42, wherein one or both of the first extension and the second extension is separable from the main region, and wherein the second side of the separated first extension or the second side of the separated second extension is removably attachable to the patient.

44. The fixation structure of any one of claims 36 to 43, wherein the first sensor componentry is at least partially flexible.

45. The fixation structure of any one of claims 36 to 44, wherein the first side of the main region of the body comprises a patient attachment.

46. The fixation structure of claim 45, wherein the or each attachment comprises an adhesive and/or the patient attachment comprises an adhesive.

47. The fixation structure of claim 46, wherein the adhesive is moisture permeable and/or porous.

48. The fixation structure of claim 46 or 47, wherein the adhesive is dermatologically sensitive and selected from any one or more of: a hydrocolloid-based adhesive material, a zinc oxide-based adhesive material, a silicone-based adhesive material, and/or a hydrogel-based adhesive material.

49. The fixation structure of any one of claims 46 to 48, wherein the first sensor componentry is located between the body and the patient attachment, is at least partly encapsulated within the patient attachment or is located between the body and the fixing element.

50. The fixation structure of any one of claims 36 to 48, wherein fixing element is positioned in a first region of the second side of the body and the first sensor componentry is at least partially positioned in a second region of the second side of the body.

51. The fixation structure of any one of claims 36 to 50, wherein the first sensor componentry forms a unitary structure with the fixing element and/or the first sensor componentry is embedded within the fixing element.

52. The fixation structure of any one of claims 36 to 51, wherein the first sensor componentry is located between at least part of the first extension and the main region when the first extension attaches to the second side of the main region.

53. The fixation structure of any one of claims 44 to 49, wherein the first sensor componentry is located at least partly between the main region and the patient attachment.

54. The fixation structure of any one of claims 36 to 51, wherein the first sensor componentry is located at least partly on the first extension.

55. The fixation structure of claim 43, wherein the first sensor componentry is located at least partly between the first extension and the attachment of the first extension, is at least partly encapsulated within the attachment of the first extension or is located at least partly between the first extension and the fixing element.

56. The fixation structure of any one of claims 39 to 44, comprising a second sensor componentry that is located on the second extension, at least partly located between the second extension and the attachment of the second extension, at least partly encapsulated within the attachment of the second extension, or is located at least partly between the second extension and the fixing element.

57. The fixation structure of claim 56, wherein the first sensor componentry and second sensor componentry comprise a sensing circuit.

58. The fixation structure of claim 57, wherein the first sensor componentry and second sensor componentry sense different parameters.

59. The fixation structure of claim 57 or 58, wherein the first sensor componentry and second sensor componentry sense the same parameter or linked parameters, and wherein the use of the first sensor componentry and second sensor componentry may enable more accurate readings and/or the detection of measurement errors and/or a faulty sensor componentry.

60. The fixation structure of any one of claims 36 to 59, wherein the first sensor componentry is provided in or on a multi-layer arrangement.

61. The fixation structure of claim 60, wherein the body comprises a plurality of layers and forms at least part of the multi-layer arrangement.

62. The fixation structure of claim 61, wherein the first sensor componentry is provided on a plurality of the layers of the body.

63. The fixation structure of claims 36 to 62, further comprising a wireless module.

64. The fixation structure of claim 63, wherein the wireless module comprises an antenna that transmits data sensed by the first sensor componentry and/or a receiver to receive data and/or the wireless module comprises a transceiver to transmit and receive data.

65. The fixation structure of claim 63 or 64, wherein the wireless module is positioned adjacent to the first sensor componentry.

66. The fixation structure of any one of claims 63 to 65, wherein the first sensor componentry comprises the wireless module.

67. The fixation structure of any one of claims 63 to 66, wherein the wireless module transmits and/or receives data using a wireless protocol, such as Bluetooth, BLE, RFID, ANT+, Wi-Fi or near-field communication (NFC).

68. The fixation structure of any one of claims 63 to 67, wherein the wireless module is configured to receive a prompt from an external device to transmit data and to transmit data in response to said prompt.

69. The fixation structure of any one of claims 36 to 68, wherein the first sensor componentry is powered wirelessly.

70. The fixation structure of claim 69, wherein the first sensor componentry comprises a receiving coil that receives a power from an emitting coil.

71. The fixation structure of any one of claims 36 to 70, wherein the first sensor componentry receives power from a battery, or a dry cell battery, such as a zinc-carbon cell, an alkaline cell, a lithium cell, a mercury cell, a silver-oxide cell, a nickel-cadmium cell a lithium-ion cell or nickel-metal hydride cell.

72. The fixation structure of any one of claims 64 to 68, wherein the wireless module is powered via the same source of energy as the first sensor componentry.

73. The fixation structure of any one of claims 36 to 72, wherein the sensor componentry receives power from an electrochemical cell.

74. The fixation structure of claim 73, wherein said electrochemical cell comprises a fuel cell and/or a biofuel cell.

75. The fixation structure of claim 73 or 74 wherein the electrochemical cell comprises an enzymatic biofuel cell.

76. The fixation structure of any one of claims 73 to 75, wherein the electrochemical cell comprises a fuel and at least one enzyme selected at least in part based on the fuel.

77. The fixation structure of claim 76, wherein the fuel comprises a hydrocarbon-based fuel or substrate.

78. The fixation structure of any one of claims 37 to 77, wherein the body is formed from at least partially porous material.

79. The fixation structure of any one of claims 34 to 78, wherein at least part of the fixation structure is breathable.

80. The fixation structure of any one of the preceding claims, wherein the body is formed from a material with a moisture vapour transmission rate of greater than 0 g/m²/24 h.

81. The fixation structure of claim 80, wherein the body is formed from a material with a moisture vapour transmission rate of at least 100 g/m²/24 h.

82. The fixation structure of claim 81, wherein the body is formed from a material with a moisture vapour transmission rate of at least 400 g/m²/24 h.

83. The fixation structure of any one of claims 37 to 82, wherein the fixing element is a mechanical fastener configured to attach to the patient interface.

84. The fixation structure of any one of claims 37 to 83, wherein the first sensor componentry senses at least one parameter of the patient.

85. The fixation structure of claim 84, wherein the parameter is selected from any one or more of: temperature, skin colour, patient movement, CO₂ concentration in the patient's blood (PCO₂), saturation of peripheral oxygen (SPO₂), heart rate, heart rhythm, electrical activity and/or respiratory rate.

86. The fixation structure of any one of claims 37 to 85, wherein the first sensor componentry comprises a temperature sensor, a motion sensor, an electrocardiogram (ECG) sensor and/or at least one LED.

87. The fixation structure of any one of claims 37 to 86, wherein the first sensor componentry comprises a film that changes colour in response to a change in a parameter of the patient and wherein the film is a thermochromic film that changes colour in response to a change of temperature of the patient or changes colour to reflect a detected CO₂ concentration in the patient's blood (PCO₂).

88. The fixation structure of any one of claims 37 to 87, wherein the first sensor componentry comprises a colour sensor configured to detect a colour of the skin of the patient and/or a pulse oximeter sensor that determines a saturation of peripheral oxygen (SPO₂) in the blood of the patient.

89. The fixation structure of any one of claims 37 to 88, comprising an aperture, wherein a sensor of the first sensor componentry is located in or adjacent to the aperture such that the sensor may sense a parameter of or through the skin of the patient when the fixation structure is attached to the patient and/or wherein the patient attachment comprises a thinned region where a thickness of the patient attachment is reduced, a sensor of the first sensor componentry being located directly adjacent to the thinned region such that the sensor may sense a parameter of or through the skin of the patient when the fixation structure is attached to the patient.

90. The fixation structure of claim 89, wherein the sensor is a colour sensor or pulse oximeter sensor.

91. A securement system for a patient interface for delivery of a breathable gas to a patient, the securement system comprising:
the fixation structure according to any one of claims 1 to 90; and
a surface of a patient interface configured to removably attach to the fixation structure.

92. A patient interface for delivering a breathable gas to a patient, the patient interface comprising:
the securement system of claim 91; and
an interface attachment element that is removably attachable to the fixing element of the fixation structure.

93. The patient interface of claim 92, comprising any one of: a nasal cannula assembly, face mask, nasal mask, oro-nasal mask, ET tube, or other breathable gas delivery structure.

94. The patient interface of claim 92 or 93, further comprising at least one wing or arm, wherein the surface of the patient interface is located on a patient facing side of the wing or arm.

95. A system for delivery of a breathable gas to a patient, comprising:
the patient interface of any one of claims 92 to 94;
a gas source; and
a tube fluidly connecting the patient interface to the gas source.

96. A system for delivery of a breathable gas to a patient, comprising:
a patient interface; and
the fixation structure according to any one of claims 1 to 90,
wherein the fixing element of the fixation structure is removably attachable to the patient interface.

97. A system for delivery of a breathable gas to a patient, comprising:
a patient interface; and
a pair of the fixation structures according to any one of claims 1 to 90,
wherein the fixing elements of each of the pair of fixation structures is removably attachable to the patient interface.

98. The system of claim 96 or 97, further comprising a naso-gastric feeding tube.

99. A patient monitoring system, comprising:
at least one patient interface for delivering a breathable gas to a patient;
at least one fixation structure of any one of claims 1 to 90, each fixation structure is removably attachable to a patient interface; and
a monitoring device that receives data sensed by the sensor componentry of each fixation structure, the monitoring device configured to display the received data to the patient and/or a caregiver of the patient.

100. The patient monitoring system of claim 99, wherein the monitoring device receives the data sensed by the sensor componentry wirelessly and wherein the system comprises an alarm that is activated by the monitoring device when the received data is above or below a threshold value or outside a predetermined range.
